(19) United States
(12) Patent Application Publication
(10) Pub. No.: US 2026/0207772 A1
(43) Pub. Date: Jul. 23, 2026

(54) CATIONIC CROSS-LINKED VESICLES

(71) Applicant: Bracco Suisse SA, Cadempino (CH)

(72) Inventors: Raphaël DE MATOS, Lausanne (CH); Riccardo GALBUSERA, Missaglia (IT); Anthony CLOUET, Prévessin-Moëns (FR); Samir CHERKAOUI, Feigeres (FR)

(73) Assignee: Bracco Suisse SA, Cadempino (CH)

(21) Appl. No.: 19/139,565

(22) PCT Filed: Dec. 22, 2023
(86) PCT No.: PCT/EP2023/087623

§ 371 Date: Jun. 16, 2025

(30) Foreign Application Priority Data
Dec. 22, 2022 (EP) 22216078

(51) Int. Cl. A61K 47/69; A61K 9/50; A61K 47/58; A61K 47/64

(52) U.S. Cl. CPC A61K 47/6929; A61K 9/5052; A61K 9/5089; A61K 47/58; A61K 47/6455

(57) ABSTRACT

A crosslinked vesicle comprising an outer layer and an inner core, said outer layer comprising a cationic amphiphilic peptide and said inner core comprising a fluorinated compound in liquid form, wherein said cationic amphiphilic peptide is a compound of formula (I) HB-CL-HP (I), wherein HB is a fluorinated hydrophobic block, CL is a cross-linking motif, HP is a cationic hydrophilic amino acid sequence having an alpha-helix structure and comprising at least (1) positive charge and said vesicle has a zeta potential of at least (20) mV.

### TECHNICAL FIELD

The invention generally relates to cationic cross-linked vesicles. The invention further relates to a method for the preparation of an aqueous suspension of said vesicles and its use in ultrasound mediated diagnostic and/or therapeutic treatments.

### BACKGROUND OF THE INVENTION

Ultrasound (US) imaging has become one of the most performed clinical imaging procedure. In order to increase the performance of the imaging, contrast-enhanced US (CEUS) agents, such as gas-filled microbubbles (MBs), are intravenously injected into patients. These MBs are often formed by a perfluorocarbon gas encapsulated in a lipid shell. When US frequencies are applied to these bubbles they resonate, generating a strong scattering, resulting in a contrast signal. By using the US at higher pressures, the bubbles collapse generating a shock wave that can create small pores and even disrupt cellular membranes. This phenomenon, so-called sonoporation opened a new potential field for therapeutic agents' delivery.

Despite those advantages, MBs suffer from a short in vivo lifetime and cannot accumulate into tissues due to their large size, generally between 1 and 10 μm.

Perfluorocarbon (PFC) nanodroplets (NDs) could overcome those challenges thanks to their sub-micrometric sizes and enhanced stability. These phase-change contrast agents (PCCA) or perfluocarbon nanodroplets are traditionally stabilized by surfactants, lipids, proteins, or polymers and can be vaporized into echogenic MBs through focused US irradiation.

WO2019/023706 discloses PFC-NDs (i.e. nanopeptisomes) having a perfluorocarbon liquid core containing a cargo, e.g. a therapeutically active agent, and a plurality of amphiphilic peptides surrounding the perfluorocarbon core, wherein said amphiphilic peptides comprise a fluorinated hydrophobic block, such as a fluorinated hydrophobic amino acid sequence, a cross-linking motif, and a hydrophilic amino acid sequence, such as a targeting motif. The disclosed nanopeptisomes are characterized by a zeta potential lower than 15 mV. Nanopeptisomes are obtained through a solvent-exchange procedure in which water is slowly added to an organic emulsion of peptide and PFC, ultimately leading to the spontaneous assembly of the peptide at the surface of PFC nanodroplets.

Up to now, according to Applicants' knowledge, such PFC-NDs stabilized by said amphiphilic peptides have not been obtained with an overall positive charge higher than 20 mV.

The Applicant has now prepared a cationic cross-linked vesicle comprising an inner core and an outer shell, said inner core comprising a fluorinated compound in liquid form and said outer shell comprising a cationic amphiphilic peptide having specific structural properties and positive charges and wherein said cross-linked vesicle has a zeta potential higher than 20 mV.

Furthermore, the Applicant has prepared cationic cross-linked vesicles stabilized by a mixture of amphiphilic peptides which unexpectedly endowed to significantly high zeta potential.

### SUMMARY OF THE INVENTION

An aspect of the invention relates to a cross-linked vesicle comprising an outer layer and an inner core, said outer layer comprising a cationic amphiphilic peptide and said inner core comprising a fluorinated compound in liquid form, wherein said cationic amphiphilic peptide is a compound of formula (I)

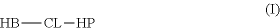

wherein
HB is a fluorinated hydrophobic block,
CL is a cross-linking motif
HP is a cationic hydrophilic amino acid sequence having an alpha-helix structure and comprising at least 1 positive charge, and

Wherein said vesicle has a zeta potential of at least 20 mV.

Preferably HP comprises at least 2 positive charges, more preferably at least 3 positive charges, still more preferably at least 4 positive charges, still more preferably at least 5 positive charges, up to 40.
In a preferred embodiment HB is a fluorinated hydrophobic amino acid sequence HB′.

Preferably HB′ comprises three consecutively connected pentafluoro-phenylalanine residues at a terminal thereof and is a compound of formula IV

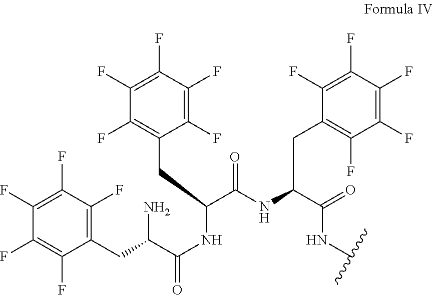 [SMILES: *NC(=O)[C@H](Cc1c(F)c(F)c(F)c(F)c1F)NC(=O)[C@H](Cc1c(F)c(F)c(F)c(F)c1F)NC(=O)[C@@H](N)Cc1c(F)c(F)c(F)c(F)c1F]

In another preferred embodiment, the cross-linking motif CL comprises a cysteine.

Preferably CL comprises the amino acid sequence GGGCCGG.

In a further preferred embodiment, said cationic amphiphilic peptide is selected from the group consisting of

H₂N-F_{F}F_{F}F_{F}GGGCCGGKGPLSSIFSRIGDP-NH₂
or
H₂N-F_{F}F_{F}F_{F}GGGCCGGKGAGYLLGKLLOOLAAAALOOLL-NH₂.

In another embodiment, said outer layer further comprises an additional amphiphilic peptide having formula VI

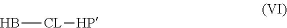

wherein
   HB is a fluorinated hydrophobic block,
   CL is a cross-linking motif and
   HP′ is a hydrophilic amino acid sequence.

Preferably, said additional amphiphilic peptide is a cationic amphiphilic peptide, wherein HP′ is a cationic hydrophilic amino acid sequence comprising at least one positive charge.

In a preferred embodiment, said additional amphiphilic peptide is selected from the group consisting of

H₂N-F_{F}F_{F}F_{F}GGGCCGGKGYGRKKRRQRRR-NH₂,
H₂N-F_{F}F_{F}F_{F}GGGCCGGKGPKKKRKV-NH₂
or
H₂N-F_{F}F_{F}F_{F}GGGCCGKGAGA-NH₂.

In another embodiment, said fluorinated compound is a perfluorocarbon selected from perfluoropentane, perfluorohexane, or a mixture thereof.

Preferably said vesicle has a zeta potential of at least 30 mV.

Another aspect relates to an aqueous suspension comprising a plurality of cross-linked vesicles as above defined.

Preferably said vesicles are calibrated cross-linked vesicles having a z-average diameter comprised between 100 nm and 1000 nm and a polydispersity lower than 0.2.

A further aspect relates to a method for the preparation of an aqueous suspension comprising a plurality of cross-linked vesicles as defined above, comprising the steps of:
a) Preparing an aqueous phase comprising a cationic amphiphilic peptide, wherein the pH of said aqueous phase is of 4 or lower;
b) Preparing an organic phase, comprising a fluorinated compound;
c) Injecting said aqueous phase in a first inlet and said organic phase in a second inlet of a microfluidic cartridge, thereby mixing said aqueous phase and said organic phase in a mixing portion of the microfluidic cartridge, wherein the operating pressure into said microfluidic cartridge is lower than 7000 kPa, to obtain an aqueous suspension of vesicles;
d) Collecting the aqueous suspension of cross-linkable vesicles from an exit channel of the microfluidic cartridge;
f) Diluting the aqueous suspension of cross-linkable vesicles, and
e) Cross-linking the cationic amphiphilic peptides to obtain an aqueous suspension of cross-linked vesicles.

Another aspect relates to an aqueous suspension comprising a plurality of cross-linked vesicles as above defined for use in a diagnostic and/or therapeutic treatment.

An aspect relates to an assembly comprising a cross-linked vesicle as above defined and a cargo molecule, wherein said cargo molecule is electrostatically bound to the outer layer of said cross-linked vesicle.

A further aspect relates to a cationic amphiphilic peptide of formula (I)

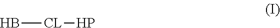

wherein
HB is a fluorinated hydrophobic block,
CL is a cross-linking motif and
HP is a cell penetrating peptide or a nuclear localization sequence having an alpha-helix structure and comprising at least 1 positive charge.

### FIGURES

FIG. 1: secondary structure prediction obtained using ColabFold (ColabFold v1.5.2-patch). Panels a) and b) show two examples of amino acid sequences arranged in an alpha helix secondary structure, namely Pres2 (PLSSIFSRIGDP) and Pepfect14 (AGYLLGKLLOOLAAAALOOLL). Panels c) and d) shows two examples of amino acid sequences arranged in a randomly-organized secondary structure, namely TAT (YGRKKRRQRRR) and SV40 (PKKKRKV).

### DETAILED DESCRIPTION OF THE INVENTION

The present invention mainly relates to a cationic cross-linked vesicle having an inner core comprising a fluorinated compound in liquid form and outer shell comprising a cationic amphiphilic peptide of formula (I)

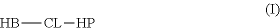

wherein
   HB is a fluorinated hydrophobic block,
   CL is a cross-linking motif
   HP is a cationic hydrophilic amino acid sequence having an alpha-helix structure and comprising at least 1 positive charge, preferably at least 5 positive charges and

said vesicle has a zeta potential of at least 20 mV, preferably at least 30 mV, more preferably at least 40 mv, up to 100 mV.

The Applicant has observed that using cationic amphiphilic peptides comprising a cationic hydrophilic amino acid sequence characterized by specific structural properties, namely an alpha-helix structure, combined with at least a positive charge, unexpectedly allowed to obtain cross-linked vesicles with a substantially high overall positive charge, e.g. zeta potential >20 mV.

Furthermore, cross-linked vesicles having an outer shell stabilized by a mixture of two or more cationic amphiphilic peptides, said mixture comprising at least one cationic hydrophilic amino acid sequence characterized by an alpha-helix structure and comprising at least a positive charge, are surprisingly characterized by significantly higher zeta potential than cross-linked vesicles stabilized by a single cationic amphiphilic peptide. Moreover, the Applicant observed that this enhanced zeta potential was associated to an improved adsorption of a cargo molecule on said outer shell.

### Definitions

The term “vesicle” indicates an assembly comprising an outer layer and an inner core, said outer layer comprising a cross-linkable amphiphilic peptide and said inner core comprising a fluorinated compound in liquid form, e.g. a perfluorocarbon. In said vesicles, the cross-linkable amphiphilic peptide molecules are oriented in such a way that the hydrophobic portions of the peptide are located at a surface of the fluorinated compound of the inner core.

According to the present invention said vesicle is a cationic vesicle bearing positive charges.

The expression “cross-linkable amphiphilic peptide” refers to any amphiphilic peptide comprising cross-linkable moieties that can potentially be covalently linked to each other through a cross-linking reaction. Suitable examples of cross-linkable moieties are cross-linkable aminoacids such as cysteine residues, that can be intermolecularly connected to an adjacent cysteine residue via disulfide cross-linking groups (—S—S—).

In a preferred embodiment, said cross-linkable moieties are cross-linkable amino acids, preferred being cysteine amino acids comprised in adjacent cross-linkable amphiphilic peptides.

According to the present invention, said cross-linkable amphiphilic peptides are preferably cationic amphiphilic peptides, comprising in their sequence at least one positive charge.

In the present description and claims, the expression “cross-linking reaction” indicates the process of forming covalent bonds between cross-linkable moieties comprised in adjacent cross-linkable amphiphilic peptides in order to bind cross-linkable amphiphilic peptides molecules together.

The term “cross-linkable vesicle” indicates an assembly comprising an outer layer and an inner core, said outer layer comprising a cross-linkable amphiphilic peptide and said inner core comprising a fluorinated compound in liquid form, wherein said cross-linkable amphiphilic peptide is not bonded intermolecularly to an adjacent amphiphilic peptide. As stated above, the cross-linkable amphiphilic peptides forming the outer layer of the cross-linkable vesicles comprise cross-linkable moieties that can potentially be covalently linked to each other.

The term “cross-linked vesicle” indicates an assembly comprising an outer layer and an inner core, said outer layer comprising a cross-linkable amphiphilic peptide and said inner core comprising a fluorinated compound in liquid form, wherein said cross-linkable amphiphilic peptide is covalently linked to an adjacent cross-linkable amphiphilic peptide through the cross-linking of the cross-linkable moieties. For instance, when the cross-linkable amphiphilic peptide includes a cysteine residue, said cysteine residue may be intermolecularly connected to an adjacent cysteine residue via disulfide cross-linking groups (—S—S—).

As mentioned above, according to the present invention said vesicle is a cationic vesicle either as cross-linkable vesicle or as cross-linked vesicle.

Preferably the vesicles of the present invention are cationic cross-linked nanodroplets, wherein the term nanodroplets refers to vesicles having a z-average diameter comprised between 100 nm and 1000 nm.

The expression “calibrated” refers to a population of cationic (per)fluorocarbon-filled vesicles as above defined (either cross-linkable or cross-linked), wherein said vesicles have a z-average diameter comprised between 100 nm and 1000 nm and a polydispersity lower than 0.25.

Generally in the state of the art, the term “calibrated” is also indicated as “size-controlled”, “uniform-sized droplets”, “monodisperse(d)” or “monosize(d)”.

In the present invention said calibrated cationic cross-linked vesicles are preferably obtained through microfluidic technique.

The expression “calibrated cationic cross-linked vesicles” indicates an aqueous suspension comprising a plurality of calibrated (per)fluorocarbon-filled cross-linkable vesicles as above defined, wherein said vesicles have a zeta potential of at least 20 mV, preferably obtained through microfluidic technique. A suspension of calibrated (per)fluorocarbon cross-linkable vesicles has not been yet submitted to any procedure aiming at inducing the cross-linking of the cross-linkable amphiphilic peptides forming the outer layer of the vesicles.

The expression “calibrated (per)fluorocarbon cross-linked vesicles” indicates an aqueous suspension of calibrated (per)fluorocarbon-filled cross-linked vesicles as above defined, said suspension being obtained through microfluidic technique. After the collection from the microfluidic cartridge, e.g. within 5 minutes, an aqueous suspension of calibrated (per)fluorocarbon cross-linkable vesicles can be submitted to a procedure aiming at inducing the cross-linking of the cross-linkable amphiphilic peptides forming the outer layer of the vesicles. As result, an aqueous suspension of calibrated (per)fluorocarbon cross-linked vesicles is obtained.

### Cationic Amphiphilic Peptide

The expression cationic amphiphilic peptide refers to a cross-linkable amphiphilic peptide, as above defined, comprising at least 1 positive charge in its sequence.

Said amphiphilic peptide is capable of assembling at the surface of a liquid fluorinated compound to form a cross-linked vesicle.

According to the present invention, the expression “cationic amphiphilic peptide” refers to a peptide of formula I

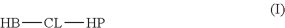

wherein HB is a fluorinated hydrophobic block, CL is a cross-linking motif and HP is a cationic hydrophilic amino acid sequence.

The cationic amphiphilic peptide has a molecular weight in the range of about 1000-5000 daltons, wherein the cationic amphiphilic peptide comprises from 5 to 50 amino acids residues, preferably from 5 to 40, more preferably from 5 to 35, wherein at least two of the amino acid residues are consecutively linked to each other in a chain by a peptide bond.

Cationic amphiphilic peptides can be synthesized using techniques known to one of ordinary skill in the art, such as, but not limited to, solid-phase synthesis, liquid-phase synthesis, recombinant methodologies polymerization, and conjugation methods.

As used herein, the term “fluorinated hydrophobic block” refers to a covalently linked chain of monomer residues forming a fluorinated hydrophobic homopolymer or copolymer. The monomeric units which form the fluorinated hydrophobic polymer may each be fluorinated according to embodiments, or some, or one, of the monomeric units is fluorinated such that at least one or more of the monomer residues of the fluorinated hydrophobic polymer is fluorinated.

According to an embodiment, the cross-linkable amphiphilic peptide does not include lipids.

According to another embodiment, the fluorinated hydrophobic polymer includes a hydrophobic amino acid sequence wherein the amino acids of the hydrophobic amino acid sequence have non-polar side chains.

According to an embodiment, the fluorinated hydrophobic polymer includes one or more synthetic non-amino acid monomeric units wherein at least one of the monomeric units is fluorinated such that at least one of the monomer residues of the fluorinated hydrophobic polymer is fluorinated. Non limiting examples of synthetic monomeric units which can be fluorinated and reacted to form a fluorinated hydrophobic polymer include methyl methacrylate, lactic acid, glycolic acid and olefins such as ethylene, propylene, styrene.

In one embodiment, the cross-linkable amphiphilic peptide is a compound of Formula (II):

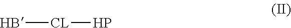

wherein HB′ is a fluorinated hydrophobic amino acid sequence; CL is a cross-linking motif; and HP is a hydrophilic amino acid sequence.

Preferably HP is a hydrophilic amino acid sequence wherein the C-terminal amino acid is amidated (—NH₂) or hydroxylated (—OH).

In another embodiment, the cross-linkable amphiphilic peptide is a compound of Formula (III):

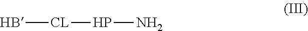

wherein HB′ is a fluorinated hydrophobic amino acid sequence; CL is a cross-linking motif; and HP is a hydrophilic amino acid sequence wherein the C-terminal amino acid is amidated.

As used herein, the term “hydrophobic amino acid sequence” refers to a sequence of hydrophobic amino acids having non-polar side chains, or a combination of a hydrophobic polymer and a sequence of hydrophobic amino acids having non-polar side chains.

Hydrophobic amino acids may be naturally occurring or non-natural (artificially produced). Examples of the naturally occurring hydrophobic amino acids include, but are not limited to, alanine, valine, leucine, isoleucine, proline, phenylalanine, tryptophan, cysteine, and methionine. Examples of non-natural hydrophobic amino acids may include D amino acids, as well as specific non-natural amino acids such as selenocysteine, pyrrolysine, and the like.

In the cross-linkable amphiphilic peptide, the fluorinated hydrophobic amino acid sequence may include one to ten fluorinated hydrophobic amino acids consecutively connected by peptide bonds, which may be unsubstituted or substituted with a substituent selected from —F, —Cl, —Br, —I, a C₁-C₃₀ alkyl group, a C₂-C₃₀ alkenyl group, a C₂-C₃₀ alkynyl group, a C₃-C₃₀ cycloalkyl group, a C₃-C₃₀ cycloalkenyl group, a C₆-C₃₀ aryl group, a C₇-C₃₀ arylalkyl group, but are not limited thereto. Fluorinated hydrophobic amino acids include, for example, fluorinated alanine, fluorinated valine, fluorinated leucine, fluorinated isoleucine, fluorinated proline, fluorinated phenylalanine, fluorinated tryptophan, fluorinated cysteine, fluorinated methionine, fluorinated selenocysteine and fluorinated pyrrolysine. The fluorinated hydrophobic amino acids can be D or L amino acids and can be fluorinated at any suitable position, typically replacing a hydrogen atom.

In a preferred embodiment, the fluorinated hydrophobic amino acids sequence may include a pentafluoro-phenylalanine (2,3,4,5,6-pentafluoro-L-phenylalanine and/or 2,3,4,5,6-pentafluoro-D-phenylalanine) at a terminal thereof.

In another embodiment, the fluorinated hydrophobic amino acid sequence may comprise one to ten consecutively connected pentafluoro-phenylalanine residues at a terminal thereof.

Still more preferably, the fluorinated hydrophobic amino acid sequence HB′ comprises three consecutively connected pentafluoro-phenylalanine residues at a terminal thereof and is a compound of formula IV

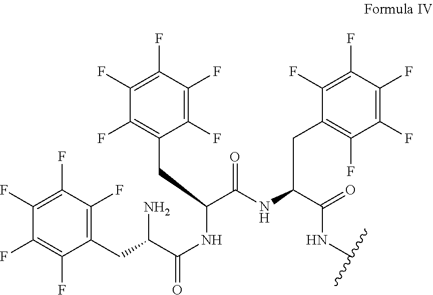 [SMILES: *NC(=O)[C@H](Cc1c(F)c(F)c(F)c(F)c1F)NC(=O)[C@H](Cc1c(F)c(F)c(F)c(F)c1F)NC(=O)[C@@H](N)Cc1c(F)c(F)c(F)c(F)c1F]

Suitable fluorinated amino acids are those described for instance in WO 2019/023707.

A combination of a hydrophobic polymer and a sequence of hydrophobic amino acids having non-polar side chains can be included in the fluorinated hydrophobic polymer wherein at least one of the monomer residues of the fluorinated hydrophobic polymer is fluorinated and/or at least one of the amino acid residues is fluorinated.

As used herein, the term “cationic hydrophilic amino acid sequence” refers to a sequence of amino acids consecutively connected by peptide bonds, wherein the hydrophilic amino acids have a polar side chain, wherein the polar side chain includes a group capable of forming a hydrogen bond with molecules of water.

In the present invention the cationic hydrophilic amino acid sequence HP can comprise both hydrophilic and hydrophobic amino acids.

Hydrophilic and hydrophobic amino acids may be naturally occurring or non-natural (artificially produced) and can be D or L amino acids.

Examples of naturally occurring hydrophilic amino acids include, but are not limited to, serine, threonine, asparagine, glutamine, histidine, arginine, lysine, aspartic acid and glutamic acid. Examples of non-natural hydrophilic amino acids include amino acids having various heterocyclic groups as a part of the side chain.

Examples of the naturally occurring and non-natural hydrophobic amino acids are those mentioned above. In the cationic amphiphilic peptide, the cationic hydrophilic amino acid sequence HP may include from 3 to 40 hydrophilic amino acids, preferably from 3 to 30, more preferably from 3 to 20, consecutively connected by peptide bonds.

According to present invention, said cationic hydrophilic amino acid sequence comprises at least one positive charge, preferably at least 2 positive charges, more preferably at least 3 positive charges, still more preferably at least 4 positive charges, still more preferably at least 5 positive charges, up to 40.

The secondary structure of said cationic hydrophilic amino acid sequence can be any suitable secondary structure, such as, but not limited to, alpha helix, n helix, beta-sheet or beta turn. Further examples of suitable secondary structure may be found in Branden, 1998.

The expression secondary structure has its conventional meaning and refers to regular, recurring arrangements in space of adjacent amino acid residues in a polypeptide chain. It is maintained by hydrogen bonds between amide hydrogens and carbonyl oxygens of the peptide backbone. The major secondary structures are α-helices and β-structures.

According to this invention, said cationic hydrophilic amino acid sequence has a secondary structure selected from the group of alpha helix, n helix, beta-sheet, beta turn or a mixture thereof.

Typically, at least 50% of the amino acid residues of said cationic hydrophilic amino acid sequence are arranged in a secondary structure, as defined above.

Alternatively, said cationic hydrophilic amino acid sequence has a randomly-organized secondary structure, e.g. a random coil structure (Smith, 1996) meaning that said sequence do not form any regular secondary structure, as defined above, and are characterized by a disordered arrangement.

In a preferred embodiment, said cationic hydrophilic amino acid sequence has an alpha-helix structure and comprises at least 1 positive charge, preferably at least 2, more preferably at least 3, more preferably at least 4, still more preferably at least 5 positive charges, up to 40. Preferably, at least 50% of the amino acid residues of said cationic hydrophilic amino acid sequence are arranged in an alpha helix.

In a preferred embodiment, said cationic hydrophilic amino acid sequence HP is selected from Pres2 (PLSSIFSRIGDP) and PepFect14 (AGYLLGKLLOOLAAAALOOLL).

The secondary structure of an amino acid sequence can be essentially predicted from its primary structure by using suitable bioinformatics methods, such as ColabFold (Mirdita, 2022).

In other words, by using said bioinformatic methods is possible to predict whether a given amino acid sequence tend to form a defined secondary structure, such as alpha helices and beta sheets, or whether it will tend to arrange in a randomly-organized secondary structure.

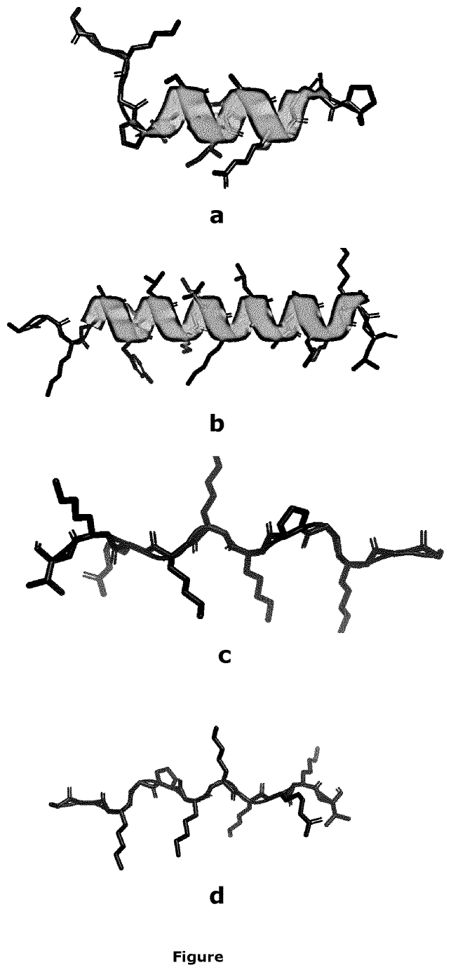

Suitable examples of secondary structure prediction obtained by using ColabFold (ColabFold v1.5.2-patch) are given by FIG. 1. FIG. 1a and 1b display two cationic hydrophilic amino acid sequences, as defined in this invention, namely Pres2 and PepFect14, arranged in an alpha helix structure, while FIGS. 1c and 1d shows two examples of hydrophilic amino acid sequences, namely TAT and SV40, arranged in a randomly-organized structure.

In a preferred embodiment, said cationic hydrophilic amino acid sequence is selected from a cell penetrating peptide (CPP) or a nuclear localization sequence (NLS).

The expression cell penetrating peptide indicates a class of short peptides with 5-30 amino acids long, preferably positively charged, that can penetrate biological membrane and deliver a wide variety of cargos into cells. A comprehensive review about CPP can be found in Derakhshankhah, 2018.

Suitable example of CPP can be found in the database CPPsite 2.0 (http://crdd.osdd.net/raghava/cppsite/).

The expression nuclear localization sequence indicates a class of short peptides based on lysine-, arginine- or proline-rich motifs that can be transported to the nucleus through the nuclear pore complex, which is a multimeric complex containing 50-100 different proteins. NLSs can be further divided into monopartite and bipartite signals, which consist, respectively, of one or two clusters of four or more basic amino acids (Lu, 2021).

In the present description and claims, the expression “cross-linking motif” refers to a cross-linkable moiety, comprised in the cross-linkable amphiphilic peptide, that can potentially be covalently linked to another cross-linkable moiety through a cross-linking reaction. Examples of cross-linking motif are sulfhydryl cross-linkers, UV cross-linkers, aza-benzenes, photosensitive cross-linkers, such as azides or benzophenones, nitriles, pH-sensitive cross-linkers, or enzymatic cross-linkers and click-chemistry-based cross-linkers (e.g. a cross-linking motif comprising an azide group able to react with an alkyne-containing cross-linking motif via click chemistry reactions, such as the copper-catalyzed azide alkyne cycloaddition).

In a preferred embodiment, the “cross-linking motif” is an amino acids sequence comprising at least one cross-linkable amino acid residues that can potentially be covalently linked to corresponding cross-linkable amino acid residues through a cross-linking reaction. The cross-linking motif may comprise from 1 to about 40 amino acid residues, preferably from 3 to 30, more preferably from 3 to 20.

In the cross-linking motif said cross-linkable amino acid residues may be at any position in the sequence and can be naturally occurring amino acids and/or non-naturally occurring amino acids.

An example of a naturally occurring amino acid able to cross-link with a corresponding cross-linkable amino acid residue is cysteine.

Non-naturally occurring amino acids may be obtained through structure functionalization of naturally occurring amino acids providing the ability to bind to a naturally occurring or non-naturally occurring amino acid in the crosslinking motif of an adjacent cross-linkable residue.

In an embodiment, the cross-linking motif comprises a cysteine.

In a further embodiment, the cross-linking motif comprises a cysteine and a glycine.

In a preferred embodiment, the cross-linking motif comprises the amino acid sequence GGGCCGG, wherein G is glycine and C is cysteine.

In an embodiment, the degree of cross-linking of the cross-linkable amphiphilic peptide molecules is higher than 80%, preferably at least 85%, more preferably at least 90%, still more preferably at least 95%. Preferably the degree of cross-linking is 100%.

The expression “degree of cross-linking” refers to the total number of cross-linked amino acid residues, e.g. cysteines, that interconnect adjacent cross-linkable amphiphilic peptides. The degree of cross-linking is generally expressed in percent and can be measured using a colorimetric disulfide formation assay.

In the present invention, preferred are cationic amphiphilic peptides comprising three pentafluoro-phenylalanine (F_{f}) residues at the N-terminus. C-terminal to this fluorinated domain is a cysteine containing crosslinking motif (GGGCCGG).

In a preferred embodiment, the cationic amphiphilic peptide is a compound of formula V

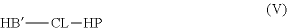

wherein
   HB′ is a fluorinated hydrophobic amino acid sequence comprising a pentafluoro-phenylalanine,
   CL is a is a cross-linking motif comprising GGGCCGG, where G is glycine and C is cysteine and
   HP is a cationic hydrophilic amino acid sequence having an alpha-helix structure and comprising at least 1 positive charge.

In a further embodiment, the cationic amphiphilic peptide is selected from the group consisting of

H₂N-F_{F}F_{F}F_{F}GGGCCGGKGPLSSIFSRIGDP-NH₂ (AP-PRES2)
or
H₂N-F_{F}F_{F}F_{F}GGGCCGGKGAGYLLGKLLOOLAAAALOOLL-NH₂
(AP-PEPFECT14).

In this description and claims, the amino acid sequences are reported following the conventional one letter code, wherein for instance G is Glycine, C is Cysteine, K is Lysine, P is Proline, L is Leucine, S is Serine, F is Phenylalanine, D is Aspartic Acid, I is Isoleucine, R is Arginine O is Ornithine, A is Alanine and Y is Tyrosine.

In this description and claims, each modified amino acid Ff is pentafluoro phenylalanine (2,3,4,5,6-pentafluoro-L-phenylalanine).

### Mixture of Cationic Amphiphilic Peptides

Advantageously the Applicant has observed that using a mixture comprising two or more amphiphilic peptides, said mixture comprising at least one cationic hydrophilic amino acid sequence characterized by an alpha-helix structure and comprising at least a positive charge, surprisingly led to cross-linked vesicles characterized by a significantly high zeta potential. Moreover, the Applicant observed that this enhanced zeta potential was associated to an improved adsorption of a cargo molecule on said outer shell.

In a preferred embodiment, the invention relates to a cross-linked vesicle as above defined, wherein said outer layer further comprises an additional amphiphilic peptide having formula VI

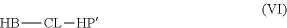

wherein
   HB is a fluorinated hydrophobic block,
   CL is a cross-linking motif and
   HP′ is a hydrophilic amino acid sequence.
   Preferably, HB and CL can be as defined above.

Preferably, said additional amphiphilic peptide is a cationic amphiphilic peptide.

According to this invention, said additional cationic amphiphilic peptide can be any suitable cationic amphiphilic peptide as defined above.

According to this invention, said hydrophilic amino acid sequence HP′ can be any hydrophilic amino acid sequence as defined above.

For instance HP′ can be selected from the group of amino acid sequences consisting of AGA, TAT (YGRKKRRQRRR) or SV40 (PKKKRKV).

Preferably HP′ is a cationic hydrophilic amino acid sequence comprising at least one positive charge, more preferably 2 or more positive charges, up to 40.

The secondary structure of HP′ can be any suitable secondary structure, such as alpha helix, n-helix, beta-sheet or beta turn as defined above.

According to this invention, HP′ has a secondary structure selected from the group of alpha helix, n-helix, beta-sheet, beta turn or a mixture thereof.

Alternatively, HP′ has a randomly-organized secondary structure.

In another embodiment, at least 50% of the amino acid residues of HP′ are arranged in a secondary structure, as defined above.

In a further embodiment, the additional amphiphilic peptide is selected from the group consisting of

H₂N-F_{F}F_{F}F_{F}GGGCCGGKGYGRKKRRQRRR-NH₂ (AP-TAT),
H₂N-F_{F}F_{F}F_{F}GGGCCGGKGPKKKRKV-NH₂ (AP-SV40),
or
H₂N-F_{F}F_{F}F_{F}GGGCCGKGAGA-NH₂ (AP-AGA).

In a preferred embodiment, HP′ has a randomly-organized secondary structure and comprises at least 5 positive charges.

### Fluorinated Compound

As described above, “cross-linked vesicle” indicates an assembly comprising an outer layer and an inner core, said outer layer comprising a cross-linkable amphiphilic peptide and said inner core comprising a fluorinated compound in liquid form that allows for activation of the vesicle upon application of ultrasound (US).

In the present description and claims, the term “fluorinated compound” refers to a group of fluorine-containing compounds derived from hydrocarbons by partial or complete substitution of hydrogen atoms with fluorine atoms, which are liquid at room temperature. Preferably the fluorinated compound is a perfluorocarbon (PFC), i.e. a fluorinated hydrocarbon where all the hydrogen atoms are substituted with fluorine atoms.

Said fluorinated compounds are selected from fluorinated compounds having a high boiling point, i.e. above room temperature (RT; 25° C.) that are in a liquid form at Standard Ambient Temperature and Pressure (SATP), namely at 25° C. and 1 atm (101.325 kPa).

Liquid fluorinated compounds are characterized by a boiling point comprised between 25° C. and 160° C. In the present invention, the fluorinated compounds are preferably characterized by a boiling point comprised between 25° C. and 100° C., still more preferably between 27° C. and 60° C.

Suitable examples of fluorinated compounds include C4-C6 fluorinate compounds, such as 1-Fluorobutane, 2-Fluorobutane, 2,2-Difluorobutane, 2,2,3,3-Tetrafluorobutane, 1,1,1,3,3-Pentafluorobutane, 1,1,1,4,4,4-Hexafluorobutane, 1,1,1,2,4,4,4-Heptafluorobutane, 1,1,2,2,3,3,4,4-Octafluorobutane, 1,1,1,2,2-Pentafluoropentane, 1,1,1,2,2,3,3,4-Octafluoropentane, 1,1,1,2,2,3,4,5,5,5-Decafluoropentane, 1,1,2,2,3,3,4,4,5,5,6,6-Dodecafluorohexane.

Suitable examples of perfluorocarbons are perfluoropentane, perfluorohexane, perlfluoroheptane, perfluorooctane, perfluorononane, perfluorodecalin, perfluorooctylbromide (PFOB), perfluoro-15-crown-5-ether (PFCE), perfluorodichlorooctane (PFDCO), perfluorotributylamine (PFTBA), perfluorononane (PFN), and 1,1,1-tris(perfluorotert-butoxymethyl)ethane (TPFBME), or a mixture thereof.

In an embodiment said perfluorocarbon is preferably perfluoropentane (PFP) (boiling point 29° C.), perfluorohexane (PFH) (boiling point 57° C.) or a mixture thereof.

### Preparation of Cationic Cross-Linked Vesicles

According to the present invention, the disclosed cationic cross-linked vesicles can be prepared by using any preparation technique suitable to manufacture calibrated nanodroplets having a Z-average diameter comprised between 100 nm and 1000 nm, preferably between 120 and 800 nm, more preferably between 150 and 400 nm.

Example of preparation techniques include sonication, homogenization, extrusion, microfluidic and microbubble condensation.

In a preferred embodiment, said cationic cross-linked vesicles is prepared by using a preparation technique suitable to manufacture calibrated nanodroplets having a polydispersity index (PDI) lower than 0.25, preferably lower than 0.20, more preferably lower than 0.15, even more preferably lower than 0.10, and a Z-average diameter comprised between 100 nm and 1000 nm, preferably between 120 and 800 nm, more preferably between 150 and 400 nm.

For instance, said preparation technique is selected from sonication, microbubble condensation or microfluidic, preferably said preparation technique is microfluidic.

In the present description and claims the expression “microfluidic technique” refers to a technology of manufacturing calibrated nanodroplets, such as the cationic cross-linked vesicle of this invention, through a microfluidic cartridge designed to manipulate fluids in channels at the microscale.

Said microfluidic technique is a bottom-up approach, that is to say that the nanodroplets are obtained by assembling molecules (e.g. amphiphilic lipid compounds and (per)fluorocarbons) into larger nanostructures (i.e. calibrated nanodroplets).

More preferably said microfluidic technique is carried out by using a mixing device such as a microfluidic cartridge equipped by a staggered herringbone micromixer or toroidal mixer.

A detailed description of a microfluidic cartridge suitable for the microfluidic process of the present invention is described in WO2022101365A1 (Bracco Suisse SA).

An aspect of the invention relates to a method for the preparation of an aqueous suspension comprising a plurality of cross-linked vesicles as defined above, comprising the steps of:
a) Preparing an aqueous phase comprising a cationic amphiphilic peptide, wherein the pH of said aqueous phase of 4 or lower;
b) Preparing an organic phase, comprising a fluorinated compound;
c) Injecting said aqueous phase in a first inlet and said organic phase in a second inlet of a microfluidic cartridge, thereby mixing said aqueous phase and said organic phase in a mixing portion of the microfluidic cartridge, wherein the operating pressure into said microfluidic cartridge is lower than 7000 kPa, to obtain an aqueous suspension of vesicles;
d) Collecting the aqueous suspension of cross-linkable vesicles from an exit channel of the microfluidic cartridge;
e) Diluting the collected aqueous suspension of cross-linkable vesicles, and
f) Cross-linking the cationic amphiphilic peptides to obtain an aqueous suspension of cross-linked vesicles.

According to a preferred embodiment, said aqueous phase further comprises an additional amphiphilic peptide, preferred being an additional cationic amphiphilic peptide.

According to the disclosed method, it is possible to obtain an aqueous suspension of calibrated cross-linkable vesicles by a single passage of the liquid phases through the microfluidic cartridge mixing portion.

### Step a) Preparing an Aqueous Phase

The “aqueous phase” typically comprises an aqueous liquid component, including, for instance, water, aqueous buffered solutions or aqueous isotonic solutions. Said aqueous phase is in liquid form.

Suitable examples of aqueous buffered solutions are phosphate-buffered saline (i.e. PBS buffer), sodium acetate buffer, tris(hydroxymethyl)aminomethane buffer (i.e. TRIS buffer) or a mixture thereof.

Suitable examples of isotonic solutions are Ringer solution, Ringer's lactate solution, saline, oral rehydration solution or a mixture thereof.

Preferably the aqueous liquid component has a pH lower than 7.0, more preferably lower than 6.5, still more preferably lower than 6.0, more preferably lower than 5.5., more preferably lower than 5, more preferably lower than 4.5, still more preferably lower than 4.0, up to e.g. 0, preferably 1.0.

Preferably the aqueous liquid component is sodium acetate buffer.

For instance, a cationic amphiphilic peptide can be admixed with an aqueous component through traditional techniques (e.g. stirring) in order to prepare the aqueous phase to be injected into the first inlet of the microfluidic cartridge.

According to a preferred embodiment, said aqueous phase further comprises an additional amphiphilic peptide, preferred being an additional cationic amphiphilic peptide.

At step a) the aqueous phase comprises a cross-linkable cationic amphiphilic peptide preferably at a concentration ranging between 0.0003 mmol/mL and 0.006 mmol/mL, more preferably between 0.0006 mmol/mL and 0.004 mmol/mL, still more preferably between 0.001 mmol/mL and 0.003 mmol/mL.

In an embodiment, at the step a) the temperature of the aqueous phase is typically lower than 25° C., preferably lower than 10° C., more preferably the temperature is about 5±2° C. Said temperature is preferably not lower than 2° C.

### Step b) Preparing an Organic Phase

The “organic phase” typically comprises an organic solvent, preferably miscible with water, including, for instance, methanol, ethanol, isopropanol, acetonitrile, DMF, DMSO and acetone. Preferably the organic solvent is ethanol. Said organic phase is in liquid form.

In the present invention the expression “organic solvent miscible with water” indicates an organic solvent capable of mixing in any ratio (e.g. any concentration) with water without separation of the two phases, i.e. forming a homogeneous solution.

For instance, C₁-C₃ alcohols, such as methanol, ethanol and propanol, are very soluble in water due to the hydrogen bonding engaging the hydroxyl groups in the alcohol molecules and the water molecules. However as the length of the hydrocarbon chain increases, the solubility in water decreases leading to a low miscibility of the two liquids that, if mixed, will form two immiscible layers.

According to a preferred embodiment, said organic phase comprises a fluorinated compound or a mixture of different fluorinated compounds dispersed in the organic solvent. Preferably the fluorinated compounds are perfluorocarbons.

Suitable examples of fluorinated compounds are those mentioned above.

As an example, a fluorinated compound in liquid form can be admixed with an organic solvent in liquid form through traditional techniques (e.g. stirring) in order to prepare the liquid organic phase to be injected into the second inlet of the microfluidic cartridge.

In a further embodiment, at step b) the organic phase comprises a fluorinated compound at a concentration ranging between 0.003 mmol/mL and 0.142 mmol/mL, more preferably between 0.011 mmol/mL and 0.085 mmol/mL, still more preferably between 0.013 mmol/mL to 0.057 mmol/mL.

In an embodiment, the organic solvent is a polar organic solvent.

The expression “polar organic solvent” has its conventional meaning in the chemical field. Solvents can be classified by their relative polarity (rp): for example, water is the most polar solvent and it is characterized by a relative polarity of 1. On the contrary non-polar solvents have low value of relative polarity, such as dimethylformamide (DMF) with a relative polarity value of 0.386.

The Applicant observed that a low or medium-low polarity of the organic solvent may negatively affect the monodispersity of the final suspension of cross-linked vesicles. For instance, the use of organic solvents having a relatively low polarity, such as dimethylformamide (rp 0.386) or isopropyl alcohol (rp 0.546), may result in vesicles size distributions with relatively high values of PDI (e.g. higher than 0.25). On the contrary, the use of organic solvents having a relatively higher polarity, such as methanol (rp 0.762) or ethanol (rp 0.654), generally allow to obtain particle size distributions with lower PDI values, indicating a good monosdispersity of the samples.

Moreover, also the cross-linked vesicles sizes may be influenced by the polarity of the organic phase. In particular, sizes were inversely proportional to the polarity of the organic phase: high polar solvents, like methanol and ethanol, led to lower sizes than low and medium-low polar solvents, like DMF and isopropyl alcohol.

In a preferred embodiment, the organic solvent is a polar organic solvent, said solvent having a polarity comprised between 0.60 and 0.80, preferably comprised between 0.63 and 0.78, still more preferably comprised between 0.65 and 0.77.

In a further embodiment, said organic solvent is selected from methanol, ethanol and mixture thereof. Preferably the organic phase is ethanol.

At step b) the temperature of said liquid organic phase is preferably lower than room temperature (25° C.), e.g. about 4° C., to avoid vaporization of fluorinated compounds having a boiling point close to 25° C.

In an embodiment, at the step b) the temperature of said liquid organic phase is typically lower than 25° C., preferably lower than 10° C., more preferably the temperature is about 5±2° C. Said temperature is preferably not lower than 2° C.

Step c) Injection into the Microfluidic Cartridge

Typically, at step c) the injection of the aqueous phase and the injection of the organic phase are carried out simultaneously.

The expression “simultaneously” indicates the simultaneous injection (i.e. co-injection) of the aqueous phase and the organic phase into the microfluidic cartridge, that is to say that the aqueous phase and organic phase are injected into two separate inlets of the microfluidic cartridge at the same time or at substantially the same time (e.g. within few seconds).

In a preferred embodiment, both aqueous and organic phases are injected into the microfluidic cartridge at a temperature suitable to avoid or substantially limit the evaporation of the fluorinated compound. For instance, after their respective preparations (i.e. step a) and step a)) both the aqueous phase and the organic phase can be stored in an ice bath (about 4° C.) before their injection into the separate inlets of the microfluidic cartridge (e.g. for 5 minutes), in order to limit the temperature increase during the time between step a), step b) and the subsequent step c).

According to the present invention, after their injections, the aqueous phase and the organic phase are directed towards a mixing device, wherein they are mixed (e.g. through laminar mixing in the case of a staggered herringbone micromixer endowing to the formation of NDs.

Typically, the operating pressure into the microfluidic cartridge is lower than 1000 psi (about 7000 kPa), preferably lower than 500 psi (about 3500 kPa), still more preferably lower than 300 psi (about 2000 kPa), still more preferably lower than 100 psi (about 700 kPa), e.g. between 10 and 90 psi.

The temperature of the mixing portion, wherein the mixing process takes place into the peculiar micro-channel geometry of the mixing portion, can be comprised between 0° C. and 25° C., preferably comprised between 0° C. and 15° C., more preferably between 0° C. and 5° C.

For instance, the microfluidic cartridge can be stored in the fridge (e.g. 4° C.), for a suitable time able to reach the desired temperature.

### Total Flow Rate (TFR) and Flow Rate Ratio (FRR)

The method of the present invention allows controlling the (per)fluorocarbon cross-linked vesicles characteristics by varying two process parameters: the Total Flow Rate and the Flow Rate Ratio.

The expression “Total Flow Rate (TFR)” refers to the total flow of both fluid streams, namely the aqueous phase and the organic phase, being pumped through the two separate inlets of the microfluidic cartridge. The unit of measurement of the TFR is mL/min.

According to an embodiment, the TFR is preferably comprised between 2 mL/min and 200 mL/min, preferably 2 and 18 mL/min, more preferably between 5 mL/min and 16 mL/min, still more preferably the TFR is 10 mL/min.

The expression “Flow Rate Ratio (FRR)” refers to the ratio between the amount of aqueous phase and the amount of organic phase flowing into the microfluidic cartridge, according to the Equation 1:

$$Flow rate ratio = \frac{volume of aqueous phase}{volume of organic phase} \qquad Eq . l$$

The volume of aqueous and organic phases can be expressed as e.g. mL.

In a preferred embodiment, the FRR (volume of aqueous phase vs. volume of organic phase) is between 1:1 to 5:1, preferably between 1:1 and 3:1, more preferably the FRR is 2:1.

In the present invention, the respective concentrations of both cross-linkable amphiphilic peptide and (per)fluorocarbon and the FRR can be purposely tuned in order to obtain a molar ratio between said cross-linkable amphiphilic peptide and said (per)fluorocarbon suitable to assure the stability of the cross-linked vesicle (i.e certain values of sizes and PDI). The molar ratio between said cross-linkable amphiphilic peptide and said (per)fluorocarbon is preferably comprised between 0.002 and 7.000. More preferably the ratio is not higher than 6.000, not higher than 5.000, not higher than 4.000, not higher than 3.000, not higher than 2.000, even more preferably not higher than 1.500. More preferably the molar ratio is not lower than 0.001, not lower than 0.004, even more preferably not lower than 0.050.

### Step e) Dilution

In an embodiment, the method of preparation further comprises a step e), which comprises diluting the aqueous suspension of calibrated cross-linkable vesicles.

In an embodiment, the step e) is carried out sequentially to step d). For example, the step e) can be performed between step e) and step f), in particular after the step d), for instance within 5 minutes from collecting the sample from the microfluidic cartridge, and before starting the cross-linking phase.

The Applicant has unexpectedly observed that a dilution step carried out after the production of the calibrated cross-linkable vesicles using a microfluidic cartridge, has a favorable effect on the initial size and initial monodispersity. Indeed, without dilution, the cross-linkable vesicles size was larger than with a dilution.

As indicated above, the expressions “initial monodispersed distribution” and “initial sizes” refer to the values of monodispersity and sizes of the calibrated (per)fluorocarbon cross-linkable vesicles composition collected from the exit channel of the microfluidic cartridge at the step d) of the disclosed method of preparation.

In the present description and claims the term “dilution” refers to the process of reducing the concentration of calibrated cross-linkable vesicles in the suspension, by adding a suitable amount of aqueous component.

A suitable amount of aqueous component corresponds to the quantity of aqueous solution necessary to reduce the concentration of the calibrated vesicles in the aqueous suspension from 2 to 10-folds.

In a preferred embodiment, the optional step e) of the present method comprises diluting the aqueous suspension of calibrated fluorocarbon vesicles from 1 to 20-folds, preferably from 2 to 10-folds, still more preferably from 3- to 8-folds e.g. about 5-fold.

Suitable aqueous components are water or oxidizing solutions as described below.

Preferably said aqueous components are oxidizing solutions, preferred being an aqueous solution of DMSO.

In a preferred embodiment, the step e) of the present method comprises diluting the suspension of calibrated cross-linkable vesicles with an aqueous solution of DMSO.

In a preferred embodiment, the concentration of DMSO in the aqueous solution is typically of at least 0.1%, preferably at least 1%, more preferably at least 1.5%. The concentration of said DMSO typically does not exceed 10%, preferably less than 5%, more preferably less than 3.5%.

Alternatively, the diluting step can be performed directly inside the microfluidic cartridge (i.e. in-line dilution), by way of an additional channel (e.g. placed between the mixing portion 103 and the exit channel 104 in FIG. 1 of WO2022101365A) suitable for diluting the calibrated fluorocarbon cross-linkable vesicles suspension with the desired aqueous component before their direction to the exit channel and before the cross-linking step e).

### Step f) Cross-Linking Step

The step f) of the method of the present invention comprises cross-linking the cross-linkable amphiphilic peptides to obtain an aqueous suspension of calibrated fluorocarbon cross-linked vesicles.

In the present description and claims, the expression “cross-linking” indicates the process of forming covalent bonds between cross-linkable moieties comprised in adjacent cross-linkable amphiphilic peptides in order to bind cross-linkable amphiphilic peptides molecules together. Preferably said cross-linkable moieties are cross-linkable amino acids comprised in adjacent cross-linkable amphiphilic peptides.

In a preferred embodiment, said cross-linkable amino acids are cysteine amino acids comprised in adjacent cross-linkable amphiphilic peptides. Said cysteine amino acids are cross-linked via disulfide cross-linking groups (—S—S—).

In a preferred embodiment, step f) comprises contacting said aqueous suspension of calibrated cross-linkable vesicles with an oxidizing source able to induce the cross-linking of the cross-linkable amphiphilic peptides to obtain an aqueous suspension of calibrated cross-linked vesicles.

The oxidizing source is contacted with the freshly prepared aqueous suspension of calibrated cross-linkable vesicles for a time sufficient to induce the cross-linking of the cross-linkable amphiphilic peptides comprised in the outer layer of the vesicles.

In an embodiment, said suitable time is typically of at least one minute, preferably at least 30 minutes, more preferably at least one hour. The time typically does not exceed 24 hours, preferably less than 18 hours, more preferably less than 15 hours.

According to another embodiment, the oxidizing source is contacted with the freshly prepared aqueous suspension of cross-linkable vesicles for a time sufficient to induce a degree of cross-linking of the cross-linkable amphiphilic peptide molecules of higher than 80%, preferably at least 85%, more preferably at least 90%. Preferably the degree of cross-linking is 100%.

Suitable examples of oxidizing sources are oxidizing solutions, oxidizing gas or a mixture thereof.

The term “oxidizing solution” indicates any aqueous solution which may cause or contribute to induce the formation of covalent bonds between cross-linkable amino acids, e.g. between the thiol groups of cysteine amino acids comprised in adjacent cross-linkable amphiphilic peptides.

Suitable examples of oxidizing solutions are water, aqueous solution of DMSO, basic solutions comprising halogens, aqueous hydrogen peroxide comprising iodine and selenide-based catalyst in presence of air.

In an embodiment, the oxidizing solution is preferably an aqueous solution of DMSO, wherein the concentration of said DMSO is typically of at least 0.1%, preferably at least 1%, more preferably at least 1.5%. The concentration of said DMSO typically does not exceed 10%, preferably less than 5%, more preferably less than 3.5%.

In a preferred embodiment, the oxidizing solution is preferably an aqueous solution of DMSO at 2.5%.

The term “oxidizing gas” indicates any gas which may induce or contribute to the formation of covalent bonds between cross-linkable amino acids, e.g. between the thiol groups of cysteine amino acids comprised in adjacent cross-linkable amphiphilic peptides.

Suitable examples of oxidizing gas are oxygen, air or suitable mixtures of gas comprising oxygen.

In a preferred embodiment, the oxidizing gas is air.

The oxidizing gas is contacted with the freshly prepared aqueous suspension of calibrated cross-linkable vesicles for a time of at least one minute, preferably at least 30 minutes, more preferably at least one hour. The time typically does not exceed 24 hours, preferably less than 10 hours, more preferably less than 3 hours.

For instance, at the end of the microfluidic process, an oxidizing gas, e.g. air, can be injected by bubbling into the oxidizing solution comprising the dialysis device including the aqueous suspension of calibrated PFC cross-linkable vesicles.

In a still more preferred embodiment, said oxidizing source comprises a mixture of an oxidizing solution and an oxidizing gas.

Preferably, said oxidizing source comprises a mixture of an aqueous oxidizing solution and air.

In a preferred embodiment, the step f) is a dialysis procedure comprising contacting an aqueous suspension of calibrated cross-linkable vesicles with an oxidizing source able to induce the cross-linking of the cross-linkable amphiphilic peptides to obtain an aqueous suspension of calibrated fluorocarbon cross-linked vesicles

Preferably said oxidizing source is an oxidizing solution as described above.

Alternatively, said oxidizing source comprises a mixture of an oxidizing solution and an oxidizing gas as described above.

In the present description, the expression “dialysis” indicates a procedure for promoting the cross-linking of the freshly prepared aqueous suspension of calibrated (per)fluorocarbon cross-linkable vesicles, through the mechanisms described above.

According to this embodiment, after the collection from the microfluidic cartridge, the aqueous suspension of calibrated cross-linkable vesicles is loaded inside a dialysis device comprising a semi-permeable membrane which is contacted with an oxidizing source, for instance by suspending said dialysis device in a large volume of an oxidizing solution, as defined above, into which is simultaneously injected an oxidizing gas.

Suitable examples of dialysis devices comprising a semi-permeable membrane can be traditional dialysis tubings or advanced dialysis devices, such as dialysis cassettes, dialysis flasks or dialysis plates. The permeability of said semi-permeable membrane is such that the oxidizing solution can contact the calibrated (per)fluorocarbon cross-linkable vesicles, loaded inside the dialysis device, consequently inducing their cross-linking. Said dialysis procedure allows efficient recovery of an aqueous suspension of calibrated (per)fluorocarbon cross-linked vesicles.

In a preferred embodiment, the step f) is a dialysis procedure comprising the steps of:
fⁱ) loading the aqueous suspension of calibrated cross-linkable vesicles inside a dialysis device comprising a semi-permeable membrane;
fⁱⁱ) contacting said loaded dialysis device with an oxidizing solution and
fⁱⁱⁱ) collecting an aqueous suspension of calibrated cross-linked vesicles from the device.

In an embodiment, at step fⁱⁱ) the oxidizing solution is an aqueous solution of DMSO at 2.5%.

In another embodiment, step fⁱⁱ) comprises contacting said dialysis device with a mixture of an oxidizing solution and an oxidizing gas. Preferably, said mixture comprises an aqueous solution of DMSO at 2.5% and air.

Preferably step fⁱⁱ) comprises contacting said dialysis device with an aqueous solution of DMSO at 2.5% for 12 hours and air for the first hour.

For instance, the aqueous suspension of calibrated cross-linkable vesicles may be loaded inside a dialysis device comprising a semi-permeable membrane and then said device can be contacted with an aqueous solution of DMSO at 2.5% for 12 hours, under constant air bubbling for the first hour.

In a still further embodiment, step fⁱⁱ) is repeated from 1 up to 5 times, preferably up to 3 times, more preferably step fⁱⁱ) is repeated 2 times.

At the end of each step eⁱⁱ), the oxidizing solution can be replaced with fresh oxidizing solution or alternatively with a different oxidizing solution.

In an embodiment each step fⁱⁱ) can be performed by contacting the dialysis device with the same oxidizing solution, e.g. aqueous solution of DMSO at 2.5%. Said oxidizing solution may be replaced with a freshly prepared solution at the beginning of each repeated step.

Alternatively, each step fⁱⁱ) can be performed by contacting the dialysis device with a different oxidizing solution. For instance, the aqueous suspension of calibrated cross-linkable vesicles may be loaded inside a dialysis device comprising a semi-permeable membrane and then said device can be contacted with an aqueous solution of DMSO at 2.5% for 12 hours, under constant air bubbling for the first hour and sequentially it can be contacted with water for 2 hours.

In some embodiments, at step fⁱⁱ) DMSO is replaced with water to reduce the amount of DMSO in the final aqueous suspension of calibrated cross-linked vesicles.

As observed by the Applicant, following the microfluidic preparation of the cross-linkable vesicles and their cross-linking as above described, the degree of cross-linking is unexpectedly high, typically the degree of cross-linking is higher than 80%, preferably at least 85%, more preferably at least 90%, still more preferably at least 95%. Preferably the degree of cross-linking is 100%.

Optional step g: Washing

In a further embodiment, the method of the invention may comprise optional step g), which comprises washing the obtained aqueous suspension of calibrated cross-linked vesicles.

According to this embodiment, after the cross-linking phase, the calibrated cross-linked vesicles are treated using suitable washing techniques.

In the present description, the term “washing” indicates any operation carried out on the freshly prepared cross-linked vesicles suspension, finalized to remove (or substantially reduce the amount of) fluorinated compound-free assemblies, or alternatively, said washing can be finalized to replace the aqueous solution in which the cross-linked vesicles are suspended at the end of the step f) of dialysis.

In the present description, the expression “fluorinated compound-free assembly” indicates an assembly comprising cross-linkable amphiphilic peptides spontaneously assembled in a particle due to hydrophobic interactions. Said assemblies may be formed during the microfluidic process and may be present in the aqueous suspension of calibrated cross-linked vesicles at the end of the preparation process, but due to their lower sizes, they contribute to forming a second population of particles, endowing to a higher value of PDI.

For example, the fluorinated compound-free assemblies are characterized by sizes comprised between 50 nm and 150 nm.

The Applicant observed that performing a washing procedure on the freshly prepared calibrated cross-linked vesicles suspension led to lower values of PDI, increasing the stability of the microfluidically-obtained composition.

Suitable washing techniques comprise, for instance, centrifugation, ultracentrifugation, filtration and decantation.

The optional step g) is carried out sequentially to step f), for example, it is carried out after the completion of the cross-linking step, e.g. within 5 minutes.

In a preferred embodiment, the washing step g) comprises the steps of:
gⁱ) centrifuging the aqueous suspension of calibrated cross-linked vesicles obtained from step e);
gⁱⁱ) separating the supernatant phase comprising fluorinated compound-free assemblies, and
gⁱⁱⁱ) adding an aqueous solution to obtain an aqueous suspension of calibrated cross-linked vesicles.

The washing in step g) can be carried out from 1 time to 10 times, preferably is performed from 2 times to 5 times, still more preferably is performed 3 times.

The duration of the centrifugation at step gⁱ is of at least 1 minute, preferably at least 3 minutes, more preferably at least 4 minutes. The duration of the centrifugation is typically lower than 10 minutes, preferably lower than 7 minutes, more preferably the duration is 5 minutes.

The temperature at which is performed the centrifugation at step gⁱ is typically of at least 1° C., preferably at least 2° C., more preferably at least 3° C. Said temperature does not exceed 25° C., preferably is lower than 20° C., more preferably is lower than 10° C.

The rotation at which is performed the centrifugation at step gⁱ is comprised between 1000 g and 10000 g, preferably between 2000 g and 6000 g, still more preferably the rotation is 5000 g.

The aqueous solution of step gⁱⁱⁱ is preferably physiologically acceptable, comprising water (preferably sterile water), aqueous solutions such as saline (which may advantageously be balanced so that the final product for injection is not hypotonic), or solutions of one or more pharmaceutical excipients.

Suitable examples of pharmaceutical excipients are tonicity adjusting substances. Tonicity adjusting substances comprise salts or sugars, sugar alcohols, glycols or other non-ionic polyol materials (e.g. glucose, sucrose, trehalose, sorbitol, mannitol, glycerol, polyethylene glycols, propylene glycols and the like), chitosan derivatives, such as carboxymethyl chitosan, trimethyl chitosan or jellifying compounds, such as carboxymethylcellulose, hydroxyethyl starch, hydrolyzed collagen, or dextran.

Preferred is a glucose solution. The glucose aqueous solution typically comprises glucose at a concentration of at least 1%, preferably at least 2.5%, more preferably at least 3%. The glucose concentration typically does not exceed 20%, preferably is less than 10%, more preferably is less than 7%.

### Assembly of Cross-Linked Vesicles and Cargo Molecule

Advantageously, the Applicant has observed that the disclosed cationic cross-linked vesicles are able to adsorb on their positive outer layer a substantially high amount of a cargo molecule.

A further aspect of said invention relates to an assembly comprising a cross-linked vesicle as defined above, and a cargo molecule, wherein said cargo molecule is electrostatically bound to the outer layer of said cross-linked vesicle.

As used herein, the term “cargo molecule” indicates a negatively-charged molecule, such as a genetic material or a nucleic acid mimetic, e.g. a peptide nucleic acid (PNAs).

In a preferred embodiment, said cargo molecule is a genetic material.

According to the present description and claims, the expression genetic material indicates any nucleic acid-based agent, such as, but not limited to, RNA, DNA, saRNA, oligonucleotides, miRNA, siRNA, shRNA molecule and dsRNA molecule. Preferably said genetic material comprises a negative charge.

In the present invention, the disclosed cationic cross-linked vesicles can deliver a cargo molecule adsorbed to their shell, due to an electrostatic interaction between the positive charges comprised in the cationic amphiphilic peptides forming said shell and the negative charges comprised in the cargo molecule.

As an example, a cargo molecule can be admixed with an aqueous suspension of cationic cross-linked vesicles through traditional techniques (e.g. stirring) in order to obtain an aqueous suspension of assemblies, i.e. entities formed by a cross-linked vesicles binding a genetic material.

After the electrostatic binding, the zeta potential of said assemblies is in general lower than the initial positive charge of the cross-linked vesicles.

The expression “initial positive charge of the cross-linked vesicles” refers to the charge of the cross-linked vesicle measured after their preparation, for instance by zeta potential determination. According to this invention, the zeta potential of said cross-linked vesicle is higher than 20 mV, preferably higher than 30, more preferably higher than 40 mV, up to 100 mV.

For instance, the zeta potential of said assemblies is lower than 20 mV, preferably lower than 10 mV, still more preferably the zeta potential has a negative value, i.e. lower than 0 mV, up to −60 mV.

Advantageously, said cationic vesicles were able to adsorb a substantially high amount of a cargo molecule on the outer shell.

In an embodiment, the aqueous suspension of assemblies comprises at least the 35% of the amount of a cargo molecule admixed to the suspension of cationic cross-linked vesicle to form an assembly as above defined. Preferably said amount is at least 50%, more preferably at least 60%, still more preferably at least 70%, still more preferably at least 80%, up to 90%, preferably up to 95%, still more preferably up to 100%.

The Applicant unexpectedly observed that the amount of cargo molecule adsorbed on the shell of cross-linked vesicles stabilized by a mixture of cationic amphiphilic peptides was higher than the amount adsorbed on the shells of cross-linked vesicles stabilized by said cationic APs when used separately as single components.

In particular, using a mixture of two or more amphiphilic peptides, said mixture comprising at least one cationic hydrophilic amino acid sequence characterized by an alpha-helix structure and comprising at least a positive charge to stabilize the outer shell of the disclosed cationic vesicles allowed to adsorb up to 4-folds the amount of cargo molecule adsorbed by cationic vesicles comprising the same amphiphilic peptides used as single shell stabilizing material.

Another aspect of the invention relates to a method for preparing an aqueous suspension comprising a plurality of assemblies as describe above, comprising the steps of:
a) preparing an initial aqueous suspension of cross-linked vesicles as describe above;
b) mixing said initial suspension with a predetermined amount of cargo molecule and
c) obtaining an aqueous suspension of assemblies, wherein the amount of said cargo molecule electrostatically bound to the outer layer of said cross-linked vesicles is at least 35% of the amount of genetic material admixed in step b), preferably at least 50%, more preferably at least 60%, still more preferably at least 70%, still more preferably at least 80%, up to 90%, preferably up to 95%, still more preferably up to 100%.

The amount of cargo molecule in the aqueous suspension of assemblies can be expressed as a percentage of the initial amount of cargo molecule (CM) in the initial suspension by using the following equation (Equation 2):

$$\% CM in the assembles suspension = \frac{\begin{matrix}amount of CM of after \\ preparation of the assemblies suspension\end{matrix}}{\begin{matrix}amount of CM admixed to the \\ cationic vesicles suspension\end{matrix}} * 100 \qquad Eq . 2$$

Wherein:
   the expression “amount of CM after preparation of the assemblies suspension” refers to the weight (e.g. μg) of cargo molecule (CM) comprised in 1 ml of suspension of assemblies and
   the expression “amount of CM admixed to the cationic vesicles suspension” refers to the weight (e.g. μg) of cargo molecule comprised in 1 ml of initial suspension of cross-linked vesicles.

Said amount can be measured for instance by analytical methods, such as photometry (UV/Vis), fluorescence, electrophoresis, diphenylamine method, quantification by real-time PCR.

### Use

The acoustic droplet vaporization (ADV) is a phenomenon through which cross-linked vesicles can be converted into gas microbubbles upon exposure to ultrasound energy beyond the vaporization threshold.

When administered in-vivo, said cross-linked vesicles present many advantages with respect to traditional microbubbles, such as inertness, relatively low toxicity, relative stability in circulation, immiscibility in water, and low surface tension (Sheeran et al, 2011). Once vaporized, the generated microbubbles can be effectively used in either imaging or therapeutic applications with ultrasound, including sonopermeabilization, thermal ablation, blood brain barrier (BBB) disruption, multimodal imaging modalities and allow passive (due to the enhanced permeability and retention (EPR) effect in the tumor tissues) or active targeting (by incorporating targeted ligands) for localized delivery of therapeutic drugs or genes. Another potentially valuable characteristic of PFC-NDs is their possible application for novel imaging strategies such as UltraSound Super-Resolution Imaging since these agents can be activated and deactivated on demand by applying intermittent acoustic pulses.

A further aspect relates to an aqueous suspension comprising a plurality of calibrated cross-linked vesicles as above defined for use in a diagnostic and/or therapeutic treatment.

Another aspect relates to an aqueous suspension comprising a plurality of assemblies as above defined for use in a diagnostic and/or therapeutic treatment.

Diagnostic treatment includes any method where the use of the cross-linked vesicles allows enhancing the visualization of a portion or of a part of an animal (including humans) body, including imaging for preclinical and clinical research. Suitable examples of diagnostic applications are molecular and perfusion imaging, tumor imaging (EPR effect), multimodal imaging (MR-guided tumor ablation, fluorescence, sono-photoacoustic activation), US aberration correction and super-resolution imaging.

Therapeutic treatment includes any method of treatment of a patient. In preferred embodiments, the treatment comprises the combined use of ultrasounds and (per)fluorocarbons vesicles either as such (e.g. in ultrasound-mediated thrombolysis, high intensity focused ultrasound ablation, blood-brain barrier permeabilization, immunomodulation, neuromodulation, radiosensitization) or in combination with a 25 therapeutic agent (i.e. ultrasound-mediated delivery, e.g. for the delivery of a drug or bioactive compound to a selected site or tissue, such as in tumor treatment, gene therapy, infectious diseases therapy, metabolic diseases therapy, chronic diseases therapy, degenerative diseases therapy, inflammatory diseases therapy, immunologic or autoimmune diseases therapy or in the use as vaccine), whereby the presence of the vesicles may provide a therapeutic effect itself or is capable of enhancing the therapeutic effects of the applied ultrasounds, e.g. by exerting or being responsible to exert a biological effect in vitro and/or in vivo, either by itself or upon specific activation by various physical methods (including e.g. ultrasound-mediated delivery).

Another aspect of the invention relates to a cationic amphiphilic peptide of formula (I)

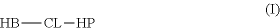

wherein
HB is a fluorinated hydrophobic block,
CL is a cross-linking motif and
HP is a cell penetrating peptide or a nuclear localization sequence, as above defined.

Preferably, HP comprises at least 2 positive charges, more preferably at least 3 positive charges, still more preferably at least 4 positive charges, still more preferably at least 5 positive charges, up to 40.
Preferably, HB is a fluorinated hydrophobic amino acid sequence HB′ as defined previously.
   Preferably, the cross-linking motif CL comprises a cysteine, more preferably comprises a comprises the amino acid sequence GGGCCGG.

The following examples will help to further illustrate the invention.

### EXAMPLES

### Materials and Methods

### Example 1

### Synthesis of Amphiphilic Peptides

Five amphiphilic peptides, namely AP-AGA, AP-TAT, AP-SV40, AP-Pepfect14, and AP-PreS2 were synthesized by FMOC solid-phase peptide synthesis (SPPS) using either H-Rink amide ChemMatrix® resin, Rink-Amide AM resin or Rink-Amide AM resin LL2, oxyma pure and DIC as coupling agent, on a PurePep Chorus Peptide Synthesizer (Gyros Protein Technologies). Resin cleavage was performed in a TFA/H₂O/EDT/TIS (94:2.5:2.5:1) solution for 5 hours. Crude peptides were then purified by preparative High Performance Liquid Chromatography (HPLC).

1. AP-AGA (H₂N-F_{F}F_{F}F_{F}GGGCCGGKGAGA-NH₂)

Purification: Solid phase: Aeris 5 μm Peptide XB-C18 100 A 250×21.2 mm. Mobile phase: 95% H₂O+0.1% TFA/5% ACN+0.1% TFA to 5% H₂O+0.1% TFA/95% ACN+0.1% TFA over 30 min.

The structural predictions of the hydrophilic amino acid sequence were performed by using AlphaFold2, through GoogleColab notebook AlphaFold2.ipynb (ColabFold 1.3.0). (https://colab.research.google.com/github/sokrypton/ColabFold/blob/main/AlphaFold2.ip ynb#scrollTo=33g5IIegij5R). The predicted secondary structure of the hydrophilic amino acid sequence AGA corresponds to a randomly-organized secondary structure.

2. AP-TAT (H₂N-F_{F}F_{F}F_{F}GGGCCGGKGYGRKKRRQRRR-NH₂)

Purification: Solid phase: Aeris 5 μm Peptide XB-C18 100 A 250×21.2 mm. Mobile phase: 90% H₂O+0.1% TFA/10% ACN+0.1% TFA to 30% H₂O+0.1% TFA/70% ACN+0.1% TFA over 30 min.

The structural predictions of the cationic hydrophilic amino acid sequence were performed by using AlphaFold2, through GoogleColab notebook AlphaFold2.ipynb (ColabFold v1.5.2-patch). (https://colab.research.google.com/github/sokrypton/ColabFold/blob/main/AlphaFold2.ip ynb#scrollTo=33g5IIegij5R).

The predicted secondary structure of the cationic hydrophilic amino acid sequence TAT (YGRKKRRQRRR) is showed in FIG. 1c and corresponds to a randomly-organized secondary structure.

3. AP-SV40 (H₂N-F_{F}F_{F}F_{F}GGGCCGGKGPKKKRKV-NH₂)

Synthesis: Pentafluorophenylalanine 19 was introduced using HATU and DIPEA as coupling agents.

Purification: Solid phase: Aeris 5 μm Peptide XB-C18 100 A 250×21.2 mm. Mobile phase: 75% H₂O+0.1% TFA/25% ACN+0.1% TFA to 50% H₂O+0.1% TFA/50% ACN+0.1% TFA over 30 min.

The secondary structure of the hydrophilic amino acid sequence SV40 (PKKKRKV) predicted using ColabFold (as indicated in Example 1 Item 2.) is showed in FIG. 1d and corresponds to a randomly-organized secondary structure.

4. AP-Pepfect14 (H₂N-F_{F}F_{F}F_{F}GGGCCGGKGAGYLLGKLLOOLAAAALOOLL-NH₂)

Purification: Solid phase: Aeris 5 μm Peptide XB-C18 100 A 250×21.2 mm. Mobile phase: 57% H₂O+0.1% TFA/43% ACN+0.1% TFA to 47% H₂O+0.1% TFA/53% ACN+0.1% TFA over 30 min.

The secondary structure of the hydrophilic amino acid sequence PepFect14 (AGYLLGKLLOOLAAAALOOLL) predicted using ColabFold (as indicated in Example 1 Item 2.) is showed in FIG. 1b and corresponds to an alpha helix structure.

5. AP-PreS2 (H₂N-F_{F}F_{F}F_{F}GGGCCGGKGPLSSIFSRIGDP-NH₂)

Purification: Solid phase: Aeris 5 μm Peptide XB-C18 100 A 250×21.2 mm. Mobile phase: 65% H₂O+0.1% TFA/35% ACN+0.1% TFA to 55% H₂O+0.1% TFA/45% ACN+0.1% TFA over 30 min

The secondary structure of the hydrophilic amino acid sequence Pres2 (PLSSIFSRIGDP) predicted using ColabFold (as indicated in Example 1 Item 2.) is showed in FIG. 1a and corresponds to an alpha helix structure.

### Example 2

### General Procedure for the Preparation of an Aqueous Suspension of Cationic Cross-Linked Vesicles Using a Microfluidic Platform

Perfluorocarbon-filled cross-linked vesicles were formulated with a NanoAssemblr™ Benchtop automated instrument from Precision Nanosystems (Vancouver, Canada) equipped with a staggered herringbone micromixer (SHM) allowing size-controlled self-assemblies. Briefly, a liquid aqueous phase comprising a cationic amphiphilic peptide was injected into the first inlet whereas a liquid organic phase composed of PFC dissolved in ethanol was injected into the second inlet of the microfluidic cartridge. Both the aqueous phase and the organic phase were placed into an ice bath at about 4° C. before the cross-linkable vesicles formulation. Microscopic characteristics of the channels are engineered to cause an accelerated mixing of the two fluid streams in a controlled fashion. The microfluidic process settings namely the Total Flow Rate (TFR, in mL/min), and the Flow Rate Ratio (FRR), were varied to control the cross-linkable vesicles characteristics.

The aqueous suspension of calibrated cross-linkable vesicles was collected from the exit channel in a Falcon vial (15 mL) and then diluted 5-times in 2.5% aqueous DMSO before being transferred to a Slide-A-Lyzer™ Dialysis Cassettes from Thermo Fisher Scientific and dialyzed against 2.5% aqueous DMSO overnight with air bubbles for the first hour. Subsequently, the suspension was further dialyzed against Milli-Q® water for hours and against aqueous glucose 5% solution for 4 hours.

At the end of the procedure, an aqueous suspension of calibrated PFC cross-linked vesicles was obtained.

The specific type and amounts of materials used in the preparations illustrated in the following examples are summarized in Table 1, wherein for all the compositions the solvent of the aqueous phase was sodium acetate and the solvent of the organic phase was ethanol. The TFR was 10 mL/min.

TABLE 1
Compositions used in the present invention

| Composition | Amphiphilic Peptide(s) (type; | | AP/PFC | |
|---|---|---|---|---|
| nr | conc) | PFC (type; conc) | Molar ratio | FRR |
| S1 | AP-PreS2; | Perfluorohexane | 0.092 | 2:1 |
| | (6.6 mg/mL) | 10 μL/mL | | |
| S2 | AP-PEPFECT14 | Perfluorohexane | 0.084 | 2:1 |
| | (8.9 mg/mL) | 10 μL/mL | | |
| S3 | AP-SV40; | Perfluorohexane | 0.077 | 2:1 |
| | (5.6 mg/mL) | 10 μL/mL | | |
| S4 | AP-TAT | Perfluorohexane | 0.040 | 1:1 |
| | (7.8 mg/mL) | 10 μL/mL | | |
| S5 | AP-TAT | Perfluoropentane | 0.040 | 1:1 |
| | (9 mg/mL) | 10 μL/mL | | |
| S6 | AP-PreS2; | Perfluoropentane | 0.080 | 2:1 |
| | (6.6 mg/mL) | 10 μL/mL | | |
| S7 | AP-PEPFECT14 | Perfluoropentane | 0.080 | 2:1 |
| | (9.5 mg/mL) | 10 μL/mL | | |
| M1 | AP-PreS2/AP-SV40 | Perfluorohexane | 0.082 | 2:1 |
| | (2.3 mg/mL)/(3.7 mg/mL) | 10 μL/mL | | |
| M2 | AP-PreS2/AP-TAT | Perfluorohexane | 0.080 | 2:1 |
| | (2.9 mg/mL)/(3.9 mg/mL) | 10 μL/mL | | |
| M3 | AP-AGA/AP-SV40 | Perfluorohexane | 0.041 | 1:1 |
| | (2 mg/mL)/(2.8 mg/mL) | 10 μL/mL | | |
| M4 | AP-AGA/AP-TAT | Perfluorohexane | 0.084 | 2:1 |
| | (2 mg/mL/(3.6 mg/mL) | 10 μL/mL | | |
| M5 | AP-PreS2/AP-SV40 | Perfluoropentane | 0.083 | 2:1 |
| | (2.6 mg/mL)/(4.3 mg/mL) | 10 μL/mL | | |
| M6 | AP-PreS2/AP-TAT | Perfluoropentane | 0.08 | 2:1 |
| | (3.3 mg/mL)/(4.5 mg/mL) | 10 μL/mL | | |
| M7 | AP-PEPFECT14/AP-SV40 | Perfluorohexane | 0.080 | 2:1 |
| | (4.2 mg/mL)/(2.9 mg/mL) | 10 μL/mL | | |
| M8 | AP-PEPFECT-14/AP-PreS2 | Perfluorohexane | 0.080 | 2:1 |
| | (4.2 mg/mL)/(2.9 mg/mL) | 10 μL/mL | | |
| M9 | AP-PEPFECT14/AP-AGA | Perfluorohexane | 0.080 | 2:1 |
| | (4.2 mg/mL)/(1.7 mg/mL) | 10 μL/mL | | |
| M10 | AP-PEPFECT14/AP-TAT | Perfluorohexane | 0.080 | 2:1 |
| | (4.2 mg/mL)/(3.9 mg/mL) | 10 μL/mL | | |
| M11 | AP-PEPFECT14/AP-SV40 | Perfluoropentane | 0.080 | 2:1 |
| | (4.8 mg/mL)/(3.3 mg/mL) | 10 μL/mL | | |
| M12 | AP-PEPFECT-14/AP-PreS2 | Perfluoropentane | 0.080 | 2:1 |
| | (4.8 mg/mL)/(3.3 mg/mL) | 10 μL/mL | | |

### Example 3

### Influence of the Nature of the Cationic Amphiphilic Peptides on the Characteristics of the Cross-Linked Vesicles

Aqueous suspensions of cationic cross-linked vesicles were prepared through the microfluidic method as described in Example 2.

In order to investigate the influence of the nature of the cationic amphiphilic peptides on the characteristics of the cross-linked vesicles (e.g. size, the PDI and Zeta Potential), different compositions were compared.

At the end of the dialysis step, the aqueous suspensions of calibrated PFC cross-linked vesicles were characterized using a Malvern Zetasizer Nano-ZS instrument (Malvern Instruments Ltd., UK) to measure the size, size distribution (PDI) and Zeta Potential (ZP). 100 μL of each suspension were diluted 10 times in aqueous KCl 1 mM for zeta potential measurements.

The characterization was performed immediately after the dialysis step (e.g. within 5 minutes) and after a one-day storage at 4° C.

### Results

TABLE 2
Sizes, PDI, ZP of Composition S1-S5 and S7 over time

| | After dialysis | | | After 1 day | | |
|---|---|---|---|---|---|---|
| | Mean | | | Mean | | |
| | diameter | | ZP | diameter | | |
| Composition | [nm] | PDI | [mV] | [nm] | PDI | ZP [mV] |
| S1 | 195 | 0.17 | 25 | 196 | 0.18 | 22 |
| S2 | 176 | 0.19 | 48 | 174 | 0.18 | 48 |
| S3 | 444 | 0.16 | 13 | 403 | 0.16 | −1 |
| S4 | 589 | 0.098 | −8.8 | — | — | — |
| S5 | 234 | 0.47 | −3.6 | 188 | 0.40 | — |
| S7 | 249 | 0.24 | 43 | 263 | 0.26 | 56 |

As inferable from Table 2 the final characteristics of the cross-linked vesicles are substantially influenced by the nature of the cationic AP comprised in their outer shell.

Cationic AP comprising a hydrophilic amino acid sequence having an alpha-helix secondary structure and at least a positive charge, such as AP-Pres2 (e.g. S1) and AP-Pepfect14 (e.g. S2 and S7), were able to form cross-linked vesicles characterized by good sizes and PDI values and having a Z potential higher than 20 mV.

On the contrary, cross-linked vesicles stabilized by cationic AP comprising a hydrophilic amino acid sequence with a randomly-organized secondary structure, such as AP-SV40 (S3) and AP-TAT (S4 and S5) were characterized by higher sizes and lower values of Z potential (<20 mV).

Moreover, the characterization carried out after one day from their preparation further demonstrated that the cross-linked vesicles stabilized by AP-Pres2 (S1) and AP-Pepfect14 (S2 and S7) were able to substantially maintain their characteristics, i.e. sizes, PDI and Z potential, over time indicating a higher stability than the cross-linked vesicles stabilized by AP-SV40 or AP-TAT.

### Example 4

### Influence of Mixture of Different Amphiphilic Peptides on the Characteristics of the Cross-Linked Vesicles

Aqueous suspensions of cationic cross-linked vesicles were prepared through the microfluidic method as described in Example 2.

In order to investigate the influence of the combination of different amphiphilic peptides on the characteristics of the cross-linked vesicles (e.g. size, the PDI and Zeta Potential), different compositions characterized by vesicles stabilized by mixture of different amphiphilic peptides were compared.

At the end of the dialysis step, the aqueous suspensions of calibrated PFC cross-linked vesicles were characterized using a Malvern Zetasizer Nano-ZS instrument (Malvern Instruments Ltd., UK) to measure the size, size distribution (PDI) and Zeta Potential (ZP). 100 μL of each suspension were diluted 10 times in aqueous KCl 1 mM for zeta potential measurements.

The characterization was performed after a one-day storage at 4° C.

TABLE 3
Sizes, PDI, ZP of Composition M1-M12 after 1-day storage

| | Mean diameter | | |
|---|---|---|---|
| Composition | [nm] | PDI | ZP [mV] |
| M1 | 192 | 0.15 | 40.4 |
| M2 | 185 | 0.14 | 48.5 |
| M3 | 251 | 0.18 | 15 |
| M4 | 238 | 0.18 | 23.9 |
| M5 | 245 | 0.23 | 48.7 |
| M6 | 244 | 0.24 | 52.9 |
| M7 | 220 | 0.17 | 63.6 |
| M8 | 254 | 0.25 | 64.3 |
| M9 | 210 | 0.10 | 58.0 |

### Results

Table 3 shows that using mixtures of APs unexpectedly led to cationic cross-linked vesicles characterized by higher zeta potential values than using the single cationic APs as such.

As inferable from the data, the increased zeta potential characterizing the cross-linked vesicles comprising a mixture of APs is higher than the zeta potential characterizing the cross-linked vesicles obtained by using said APs separately as single components (e.g. S1 and S2).

For instance, cross-linked vesicles stabilized by AP-Pres2 as such (S2), were characterized by a zeta potential of 22 mV, while cross-linked vesicles stabilized by AP-SV40 (S3) as such were characterized by a low zeta potential of 13 mV.

Surprisingly, the mixture of AP-PreS2 and AP-SV40 (M1) allowed to obtain cationic cross-linked vesicles characterized by much higher zeta potential values, i.e. higher than 40 mV.

This outcome is still more surprising considering that the cationic AP-SV40 (S3) was not able to endow to cationic cross-linked vesicles (i.e. very low ZP) when used as such (see Table 2).

The same synergic effect on the overall positive charge of the final cross-linked vesicles was confirmed using perfluoropentane (bp: 28-30° C.) as component of the core (e.g. Composition M5 and M6).

### Example 5

### Influence of the Nature of the Cationic Amphiphilic Peptides on the Adsorption of Genetic Material on the Surface of Cross-Linked Vesicles

Aqueous suspensions of cationic cross-linked vesicles were prepared through the microfluidic method as described in Example 2.

In order to investigate the influence of the nature of the cationic amphiphilic peptides on the amount of adsorbed genetic material on the shell of the cross-linked vesicles, different compositions were compared. At the end of the preparation, the aqueous suspensions of calibrated cross-linked vesicles were admixed with genetic material as reported below.

A DNAse, RNAse free 1.5 mL Eppendorf Tube® was charged with 5.2 μg of plasmid pCPG-hCMVSCEP-LucSH. 625 μL of cross-linked suspension was added slowly and the suspension was then diluted to 650 μL with aqueous glucose 5% solution, gently mixed, and the mixture was incubated for 15 min at rt.

For the measurement of the amount of pDNA in the compositions, the remaining suspension was centrifugated (10 min, 5000 rpm) at 4° C. and 20 μL of the supernatant was diluted in 180 μL of Qubit™ 1×dsDNA BR Working Solution and analyzed with a Qubit™4.

### Results

The amount of pDNA adsorbed on the shell of the cross-linked vesicles was determined according to Equation 2.

The synergic effect obtained by using mixtures of APs in comparison with using the single APs as shell components was further confirmed by results of the determination of the amount of pDNA adsorbed on the shell of the related cross-linked vesicles.

As inferable from Table 4, the amount of pDNA adsorbed on the shell of the cross-linked vesicles stabilized by a mixture of APs is significantly high and can be even higher than the amount adsorbed on the shells of cross-linked vesicles stabilized by said APs when used separately as single components.

For instance, cross-linked vesicles stabilized by a mixture of AP-PreS2 and AP-SV40 (M1) were able to adsorb about the double amount of pDNA than the cross-linked vesicles stabilized by AP-Pres2 as single component (S1).

TABLE 4
Percentage of the adsorbed pDNA

| | Percentage of pDNA |
|---|---|
| Composition | adsorbed [%] |
| S1 | 36 |
| S6 | 73 |
| S2 | 88 |
| S7 | 89 |
| S3 | 20 |
| M1 | 81 |
| M2 | 86 |
| M10 | 81 |
| M9 | 50 |
| M3 | 35 |
| M5 | 78 |
| M6 | 85 |
| M7 | 71 |
| M11 | 88 |
| M12 | 81 |

As stated above, this outcome is still more surprising considering that the cationic AP-SV40 used as single component (S3) was able to adsorb only the 20% of pDNA contacted with the aqueous suspension of cross-linked vesicles.

Furthermore, this improved amount of adsorbed pDNA was observed either in compositions comprising perfluorohexane (e.g. M1-M3) or perfluropentane (e.g. M5, M6, M11 and M12).

### Example 6

### Determination of the Cross-Linking Degree.

The determination of the cross-linking degree was performed on Composition M1 and M2. The percentage of crosslinking was measured by Ellman's assay. Briefly, a 10 mM solution of Ellman's reagent was prepared in 1 mM EDTA, 200 mM phosphate buffer pH=7.5. Ten microliters (10 μL) of each suspension and 4 μL of a solution containing Ellman's reagent were added to 140 μL of PBS buffer. The mixtures were incubated in the dark for 40 min. Absorbance was measured and the concentration was calculated using Beer-Lambert law (Eq. 2):

$$A = ε \cdot l \cdot C \qquad ( Eq . 2 )$$

wherein A is the absorbance, I is the sample chamber length (cm), E is the molar absorption coefficient (M⁻¹cm⁻¹) and C is the concentration.

In this specific example E was 14150 M⁻¹cm⁻¹.

Four microliters (4 μL) of a solution containing Ellman's reagent in 140 μL of PBS buffer were used as blank.

### Results

The degree of cross-linking was found to be higher than 90% for both composition M1 and M2 In particular M1 was characterized by a CL degree of 98.6% and M2 of 99%, demonstrating that a substantially high number of cross-linkable cysteine residues comprised in the outer shell of the cross-linked vesicles were intermolecularly connected via disulfide cross-linking groups (—S—S).

### Example 6

### Determination of Acoustic Droplet Vaporization (ADV)

The expression “Acoustic Droplet Vaporization (ADV) threshold” indicates the minimal acoustic pressure that is necessary to obtain the nanodroplets conversion into echogenic microbubbles.

The Acoustic Droplet Vaporization (ADV) threshold of the NDs prepared according to the previous examples can be determined according to conventional methodologies using B-mode imaging methods. For instance, the suspension of NDs can be vaporized while passing through the focal zone of a transducer and the acoustic pressure is increased by about 0.2 MPa each 5 s until the NDs vaporization is observed.

Nanodroplets activation was performed by focused ultrasound waves on five aligned focal points allowing the activation only within the region of interest where the acoustics pressure was highest. Pulses were emitted in burst mode at a frequency of 6 MHz, 20 cycles per pulse and at a pulse-repetition frequency (PRF) of 1 Hz.

The acoustic pressure was increased every 5 s until the observation of the NDs vaporization.

For the ADV determination, six different compositions were tested, namely S1, S2, M1 (comprising perfluorohexane) and M5-M6 (comprising perfluoropentane).

### Results

Table 5 reports the overall results obtained from the determination of the ADV thresholds. Each value is the average of three successive determinations.

TABLE 5
Determination of acoustic droplet vaporization threshold

| | Without pDNA | With pDNA |
|---|---|---|
| Composition | MPa | MPa |
| S1 | 10.27 ± 0.14 | — |
| S2 | 10.15 ± 0.09 | 10.24 ± 0.03 |
| S3 | 10.90 ± 0.13 | — |
| M1 | 10.42 ± 0.25 | 10.20 ± 0.06 |
| M5 | 7.34 ± 0.03 | — |
| M6 | 7.36 ± 0.06 | — |
| M7 | 10.05 ± 0.08 | 10.38 ± 0.05 |
| M11 | 7.46 ± 0.04 | 7.76 ± 0.24 |
| M12 | 7.47 ± 0.28 | 7.56 ± 0.16 |

Results confirmed that formulations comprising cross-linked vesicles stabilized by a single amphiphilic peptide. such as Composition S1. S2 and S3, and formulations comprising cross-linked vesicles stabilized by a mixture of amphiphilic peptides (Composition M1, M5, M6, M7, M11, M12) could be vaporized at similar acoustic pressure. The addition of pDNA to the surface of the particles did not impact the ADV threshold.

Moreover, compositions M5, M6, M11 and M12, comprising cross-linked vesicles stabilized by a mixture of APs and comprising perfluropentane in their core, were characterized by lower ADV threshold value.

### REFERENCES

1 WO2019023706A1
2 Branden, Tooze, Introduction to Protein Structure, Garland Science, 1998
3 Smith, Folding and Design, volume 1, Issue 5, Pages R95-R106 (1996)
4 Mirdita et al. Nature Methods, volume 19, pages 679-682 (2022)
5 Derakhshankhah et al. Biomed Pharmacother, volume 108, Pages 1090-1096 (2018)
6 www.cppdatabase, consulted on 22 Dec. 2022.
7 Lu et al, Cell Commun Signal.; 19:60 (2021)
8 WO2022101365 A1
9 Sheeran et al, Langmuir, 27(17), 10412-10420, (2011).

1. A cross-linked vesicle comprising an outer layer and an inner core, said outer layer comprising a cationic amphiphilic peptide and said inner core comprising a fluorinated compound in liquid form, wherein said cationic amphiphilic peptide is a compound of formula (I) 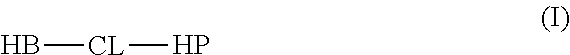
wherein
HB is a fluorinated hydrophobic block,
CL is a cross-linking motif
HP is a cationic hydrophilic amino acid sequence having an alpha-helix structure and comprising at least 1 positive charge, and
Wherein said vesicle has a zeta potential of at least 20 mV.

2. The cross-linked vesicle according to claim 1, wherein HP comprises at least 5 positive charges.

3. The cross-linked vesicle according to claim 1, wherein HB is a fluorinated hydrophobic amino acid sequence HB′.

4. The cross-linked vesicle according to claim 3, wherein said fluorinated hydrophobic amino acid sequence HB′ comprises three consecutively connected pentafluoro-phenylalanine residues at a terminal thereof and is a compound of formula IV

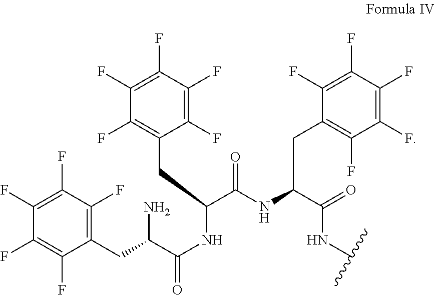 [SMILES: *NC(=O)[C@H](Cc1c(F)c(F)c(F)c(F)c1F)NC(=O)[C@H](Cc1c(F)c(F)c(F)c(F)c1F)NC(=O)[C@@H](N)Cc1c(F)c(F)c(F)c(F)c1F]

5. The cross-linked vesicle according to claim 1, wherein the cross-linking motif CL comprises a cysteine.

6. The cross-linked vesicle according to claim 5, wherein said cross-linking motif CL comprises the amino acid sequence GGGCCGG.

7. The cross-linked vesicle according to claim 1, wherein said cationic amphiphilic peptide is selected from the group consisting of
H₂N-F_{F}F_{F}F_{F}GGGCCGGKGPLSSIFSRIGDP-NH₂ [[or]]
and
H₂N-F_{F}F_{F}F_{F}GGGCCGGKGAGYLLGKLLOOLAAAALOOLL-NH₂.

8. The cross-linked vesicle according to claim 1, wherein said outer layer further comprises an additional amphiphilic peptide having formula VI 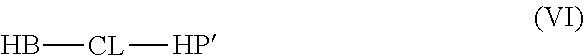
wherein
HB is a fluorinated hydrophobic block,
CL is a cross-linking motif and
HP′ is a hydrophilic amino acid sequence.

9. The cross-linked vesicle according to claim 8, wherein HP′ has a secondary structure selected from the group of alpha helix, π-helix, beta-sheet, beta turn and a mixture thereof.

10. The cross-linked vesicle according to claim 8, wherein HP′ has a randomly-organized secondary structure.

11. The cross-linked vesicle according to claim 8, wherein said additional amphiphilic peptide is a cationic amphiphilic peptide, wherein HP′ is a cationic hydrophilic amino acid sequence comprising at least one positive charge.

12. The cross-linked vesicle according to claim 11, wherein HP′ is a cationic hydrophilic amino acid sequence comprising 2 or more positive charges.

13. The cross-linked vesicle according to claim 8, wherein said additional amphiphilic peptide is selected from the group consisting of
H₂N-F_{F}F_{F}F_{F}GGGCCGGKGYGRKKRRQRRR-NH₂,
H₂N-F_{F}F_{F}F_{F}GGGCCGGKGPKKKRKV-NH₂ [[or]]
and
H₂N-F_{F}F_{F}F_{F}GGGCCGKGAGA-NH₂.

14. The cross-linked vesicle according to claim 8, wherein HP and HP′ are selected from a cell penetrating peptide and a nuclear localization sequence.

15. The cross-linked vesicle according to claim 1, wherein said fluorinated compound is a perfluorocarbon selected from perfluoropentane, perfluorohexane, and a mixture thereof.

16. The cross-linked vesicle according to claim 1, having a degree of cross-linking higher than 80%.

17. The cross-linked vesicle according to claim 1, wherein said vesicle has a zeta potential of at least 30 mV.

18. An aqueous suspension comprising a plurality of cross-linked vesicles according to claim 1.

19. The aqueous suspension according to claim 18, wherein said vesicles are calibrated cross-linked vesicles having a z-average diameter comprised between 100 nm and 1000 nm and a polydispersity lower than 0.2.

20. A method for the preparation of an aqueous suspension comprising a plurality of cross-linked vesicles according to claim 1, comprising the steps of:
a) Preparing an aqueous phase comprising a cationic amphiphilic peptide, wherein the pH of said aqueous phase is 4 or lower;
b) Preparing an organic phase, comprising a fluorinated compound;
c) Injecting said aqueous phase in a first inlet and said organic phase in a second inlet of a microfluidic cartridge, thereby mixing said aqueous phase and said organic phase in a mixing portion of the microfluidic cartridge, wherein the operating pressure into said microfluidic cartridge is lower than 7000 kPa, to obtain an aqueous suspension of vesicles;
d) Collecting the aqueous suspension of cross-linkable vesicles from an exit channel of the microfluidic cartridge;
f) Diluting the aqueous suspension of cross-linkable vesicles, and
e) Cross-linking the cationic amphiphilic peptides to obtain an aqueous suspension of cross-linked vesicles.

21. (canceled)

22. An assembly comprising a cross-linked vesicle as defined in claim 1, and a cargo molecule, wherein said cargo molecule is electrostatically bound to the outer layer of said cross-linked vesicle.

23.-28. (canceled)


SEQUENCE LISTING (15 sequences)

SEQ ID NO: 1 - protein, 7 aa, synthetic construct
  REGION 1..7: note = CROSS-LINKING MOTIF
GGGCCGG

SEQ ID NO: 2 - protein, 12 aa, synthetic construct
  HELIX 1..12
  REGION 1..12: note = PRES2
PLSSIFSRIGDP

SEQ ID NO: 3 - protein, 21 aa, synthetic construct
  HELIX 1..21
  REGION 1..21: note = PEPFECT14
AGYLLGKLLOOLAAAALOOLL

SEQ ID NO: 4 - protein, 11 aa, synthetic construct
  REGION 1..11: note = TAT
YGRKKRRQRRR

SEQ ID NO: 5 - protein, 7 aa, synthetic construct
  REGION 1..7: note = SV40
PKKKRKV

SEQ ID NO: 6 - protein, 24 aa, synthetic construct
  REGION 1..24: note = CATIONIC AMPHIPHILIC PEPTIDE
  REGION 1..3: note = Each X is pentafluoro phenylalanine (2,3,4,5,6-pentafluoro-L-phenylalanine)
  HELIX 13..24
  REGION 13..24: note = PRES2
XXXGGGCCGGKGPLSSIFSRIGDP

SEQ ID NO: 7 - protein, 33 aa, synthetic construct
  REGION 1..33: note = CATIONIC AMPHIPHILIC PEPTIDE
  REGION 1..3: note = Each X is pentafluoro phenylalanine (2,3,4,5,6-pentafluoro-L-phenylalanine)
  HELIX 13..33
  REGION 13..33: note = PEPFECT14
XXXGGGCCGGKGAGYLLGKLLOOLAAAALOOLL

SEQ ID NO: 8 - protein, 23 aa, synthetic construct
  REGION 1..23: note = CATIONIC AMPHIPHILIC PEPTIDE
  REGION 1..3: note = Each X is pentafluoro phenylalanine (2,3,4,5,6-pentafluoro-L-phenylalanine)
  REGION 13..23: note = TAT
XXXGGGCCGGKGYGRKKRRQRRR

SEQ ID NO: 9 - protein, 19 aa, synthetic construct
  REGION 1..19: note = CATIONIC AMPHIPHILIC PEPTIDE
  REGION 1..3: note = Each X is pentafluoro phenylalanine (2,3,4,5,6-pentafluoro-L-phenylalanine)
  REGION 13..19: note = SV40
XXXGGGCCGGKGPKKKRKV

SEQ ID NO: 10 - protein, 15 aa, synthetic construct
  REGION 1..15: note = CATIONIC AMPHIPHILIC PEPTIDE
  REGION 1..3: note = Each X is pentafluoro phenylalanine (2,3,4,5,6-pentafluoro-L-phenylalanine)
XXXGGGCCGGKGAGA

SEQ ID NO: 11 - protein, 24 aa, synthetic construct
  REGION 1..24: note = CATIONIC AMPHIPHILIC PEPTIDE
  REGION 1..3: note = Each X is pentafluoro phenylalanine (2,3,4,5,6-pentafluoro-L-phenylalanine)
  MOD_RES 24: note = AMIDATION
  HELIX 13..24
  REGION 13..24: note = PRES2
XXXGGGCCGGKGPLSSIFSRIGDP

SEQ ID NO: 12 - protein, 33 aa, synthetic construct; identical to SEQ ID NO: 7
  REGION 1..33: note = CATIONIC AMPHIPHILIC PEPTIDE
  REGION 1..3: note = Each X is pentafluoro phenylalanine (2,3,4,5,6-pentafluoro-L-phenylalanine)
  MOD_RES 33: note = AMIDATION
  HELIX 13..33
  REGION 13..33: note = PEPFECT14

SEQ ID NO: 13 - protein, 23 aa, synthetic construct
  REGION 1..23: note = CATIONIC AMPHIPHILIC PEPTIDE
  REGION 1..3: note = Each X is pentafluoro phenylalanine (2,3,4,5,6-pentafluoro-L-phenylalanine)
  MOD_RES 23: note = AMIDATION
  REGION 13..23: note = TAT
XXXGGGCCGGKGYGRKKRRQRRR

SEQ ID NO: 14 - protein, 19 aa, synthetic construct
  REGION 1..19: note = CATIONIC AMPHIPHILIC PEPTIDE
  REGION 1..3: note = Each X is pentafluoro phenylalanine (2,3,4,5,6-pentafluoro-L-phenylalanine)
  MOD_RES 19: note = AMIDATION
  REGION 13..19: note = SV40
XXXGGGCCGGKGPKKKRKV

SEQ ID NO: 15 - protein, 15 aa, synthetic construct
  REGION 1..15: note = CATIONIC AMPHIPHILIC PEPTIDE
  REGION 1..3: note = Each X is pentafluoro phenylalanine (2,3,4,5,6-pentafluoro-L-phenylalanine)
  MOD_RES 15: note = AMIDATION
XXXGGGCCGGKGAGA